SEQUENCE LISTING (25 sequences)

SEQ ID NO: 1 - protein, 435 aa, Artificial Sequence
  Fusion protein
MLLLVTSLLLCELPHPAFLLIPEKSDLRTVAPASSLNVRFDSRTMNLSWDCQENTTFSKCFLTDKKNRVVEPRLSNNECSCTFREICLHEGVTFEVHVNTSQRGFQQKLLYPNSGREGTAAQNFSCFIYNADLMNCTWARGPTAPRDVQYFLYIRNSKRRREIRCPYYIQDSGTHVGCHLDNLSGLTSRNYFLVNGTSREIGIQFFDSLLDTKKIERFNPPSNVTVRCNTTHCLVRWKQPRTYQKLSYLDFQYQLDVHRKNTQPGTENLLINVSGDLENRYNFPSSEPRAKHSVKIRAADVRILNWSSWSEAIEFGSDDGPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 2 - protein, 749 aa, Artificial Sequence
  Fusion protein
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 3 - protein, 441 aa, Artificial Sequence
  Fusion protein
MTSSHAMNITPLAQLALLFSTLLLPGTQALLAPTTPDAGSALNLTFDPWTRTLTWACDTAAGNVTVTSCTVTSREAGIHRRVSPFGCRCWFRRMMALHHGVTLDVNGTVGGAAAHWRLSFVNEGAAGSGAENLTCEIRAARFLSCAWREGPAAPADVRYSLRVLNSTGHDVARCMADPGDDVITQCIANDLSLLGSEAYLVVTGRSGAGPVRFLDDVVATKALERLGPPRDVTASCNSSHCTVSWAPPSTWASLTARDFQFEVQWQSAEPGSTPRKVLVVEETRLAFPSPAPHGGHKVKVRAGDTRMKHWGEWSPAHPLEAEDTRVPPSLFALEAVLIPVGTMGLIITLIFVYCWLERMPPIPPIKNLEDLVTEYQGNFSAWSGVSKGLTESLQPDYSERFCHVSEIPPKGGALGEGPGGSPCSLHSPYWPPPCYSLKPEA

SEQ ID NO: 4 - protein, 740 aa, Artificial Sequence
  Fusion protein
MDQQMALTWGLCYMALVALCWGHGVTEAEETVPLKTLQCYNDYTNHIICSWADTEDAQGLINMTLYHQLEKKQPVSCELSEELMWSECPSSHRCVPRRCVIPYTRFSITNEDYYSFRPDSDLGIQLMVPLAQNVQPPLPKNVSISSSEDRFLLEWSVSLGDAQVSWLSSKDIEFEVAYKRLQDSWEDAYSLHTSKFQVNFEPKLFLPNSIYAARVRTRLSPGSSLSGRPSRWSPEVHWDSQPGDKAQPQNLQCFFDGIQSLHCSWEVWTQTTGSVSFGLFYRPSPVAPEEKCSPVVKEPPGASVYTRYHCSLPVPEPSAHSQYTVSVKHLEQGKFIMSYNHIQMEPPTLNLTKNRDSYSLHWETQKMAYSFIEHTFQVQYKKKSDSWEDSKTENLDRAHSMDLSQLEPDTSYCARVRVKPISNYDGIWSKWSEEYTWKTDWILPMSWLRYLLLVLGCFSGFFSCVYILVKCRYLGPWLKTVLKCHIPDPSEFFSQLSSQHGGDLQKWLSSPVPLSFFSPSGPAPEISPLEVLDGDSKAVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLI

SEQ ID NO: 5 - protein, 1203 aa, Artificial Sequence
  Fusion protein
MTSSHAMNITPLAQLALLFSTLLLPGTQALLAPTTPDAGSALNLTFDPWTRTLTWACDTAAGNVTVTSCTVTSREAGIHRRVSPFGCRCWFRRMMALHHGVTLDVNGTVGGAAAHWRLSFVNEGAAGSGAENLTCEIRAARFLSCAWREGPAAPADVRYSLRVLNSTGHDVARCMADPGDDVITQCIANDLSLLGSEAYLVVTGRSGAGPVRFLDDVVATKALERLGPPRDVTASCNSSHCTVSWAPPSTWASLTARDFQFEVQWQSAEPGSTPRKVLVVEETRLAFPSPAPHGGHKVKVRAGDTRMKHWGEWSPAHPLEAEDTRVPPSLFALEAVLIPVGTMGLIITLIFVYCWLERMPPIPPIKNLEDLVTEYQGNFSAWSGVSKGLTESLQPDYSERFCHVSEIPPKGGALGEGPGGSPCSLHSPYWPPPCYSLKPEAGSGATNFSLLKQAGDVEENPGPMDQQMALTWGLCYMALVALCWGHGVTEAEETVPLKTLQCYNDYTNHIICSWADTEDAQGLINMTLYHQLEKKQPVSCELSEELMWSECPSSHRCVPRRCVIPYTRFSITNEDYYSFRPDSDLGIQLMVPLAQNVQPPLPKNVSISSSEDRFLLEWSVSLGDAQVSWLSSKDIEFEVAYKRLQDSWEDAYSLHTSKFQVNFEPKLFLPNSIYAARVRTRLSPGSSLSGRPSRWSPEVHWDSQPGDKAQPQNLQCFFDGIQSLHCSWEVWTQTTGSVSFGLFYRPSPVAPEEKCSPVVKEPPGASVYTRYHCSLPVPEPSAHSQYTVSVKHLEQGKFIMSYNHIQMEPPTLNLTKNRDSYSLHWETQKMAYSFIEHTFQVQYKKKSDSWEDSKTENLDRAHSMDLSQLEPDTSYCARVRVKPISNYDGIWSKWSEEYTWKTDWILPMSWLRYLLLVLGCFSGFFSCVYILVKCRYLGPWLKTVLKCHIPDPSEFFSQLSSQHGGDLQKWLSSPVPLSFFSPSGPAPEISPLEVLDGDSKAVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLI

SEQ ID NO: 6 - DNA, 1308 bp, Artificial Sequence
  Fusion sequence
atgcttctcctggtgacaagccttctgctctgtgagttaccacacccagcattcctcctgatcccagagaaatcggatctgcgaacagtggcaccagcctctagtctcaatgtgaggtttgactccaggacgatgaatttaagctgggactgccaagaaaacacaaccttcagcaagtgtttcttaactgacaagaagaacagagtcgtggaacccaggctcagtaacaacgaatgttcgtgcacatttcgtgaaatttgtctgcatgaaggagtcacatttgaggttcacgtgaatactagtcaaagaggatttcaacagaaactgctttatccaaattcaggaagggagggtaccgctgctcagaatttctcctgtttcatctacaatgcggatttaatgaactgtacctgggcgaggggtccgacggccccccgtgacgtccagtattttttgtacatacgaaactcaaagagaaggagggagatccggtgtccttattacatacaagactcaggaacccatgtgggatgtcacctggataacctgtcaggattaacgtctcgcaattactttctggttaacggaaccagccgagaaattggcatccaattctttgattcacttttggacacaaagaaaatagaacgattcaaccctcccagcaatgtcaccgtacgttgcaacacgacgcactgcctcgtacggtggaaacagcccaggacctatcagaagctgtcgtacctggactttcagtaccagctggacgtccacagaaagaatacccagcctggcacggaaaacctactgattaatgtttctggtgatttggaaaatagatacaactttccaagctctgagcccagagcaaaacacagtgtgaagatcagagctgcagacgtccgcatcttgaattggagctcctggagtgaagccattgaatttggttctgacgacgggcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctga

SEQ ID NO: 7 - DNA, 2250 bp, Artificial Sequence
  Fusion sequence
atggtgctggcccaggggctgctctccatggccctgctggccctgtgctgggagcgcagcctggcaggggcagaagaaaccatcccgctgcagaccctgcgctgctacaacgactacaccagccacatcacctgcaggtgggcagacacccaggatgcccagcggctcgtcaacgtgaccctcattcgccgggtgaatgaggacctcctggagccagtgtcctgtgacctcagtgatgacatgccctggtcagcctgcccccatccccgctgcgtgcccaggagatgtgtcattccctgccagagttttgtcgtcactgacgttgactacttctcattccaaccagacaggcctctgggcacccggctcaccgtcactctgacccagcatgtccagcctcctgagcccagggacctgcagatcagcaccgaccaggaccacttcctgctgacctggagtgtggcccttgggagtccccagagccactggttgtccccaggggatctggagtttgaggtggtctacaagcggcttcaggactcttgggaggacgcagccatcctcctctccaacacctcccaggccaccctggggccagagcacctcatgcccagcagcacctacgtggcccgagtacggacccgcctggccccaggttctcggctctcaggacgtcccagcaagtggagcccagaggtttgctgggactcccagccaggggatgaggcccagccccagaacctggagtgcttctttgacggggccgccgtgctcagctgctcctgggaggtgaggaaggaggtggccagctcggtctcctttggcctattctacaagcccagcccagatgcaggggaggaagagtgctccccagtgctgagggaggggctcggcagcctccacaccaggcaccactgccagattcccgtgcccgaccccgcgacccacggccaatacatcgtctctgttcagccaaggagggcagagaaacacataaagagctcagtgaacatccagatggcccctccatccctcaacgtgaccaaggatggagacagctacagcctgcgctgggaaacaatgaaaatgcgatacgaacacatagaccacacatttgagatccagtacaggaaagacacggccacgtggaaggacagcaagaccgagaccctccagaacgcccacagcatggccctgccagccctggagccctccaccaggtactgggccagggtgagggtcaggacctcccgcaccggctacaacgggatctggagcgagtggagtgaggcgcgctcctgggacaccgagtcgattccgtggctcggccacctcctcgtgggcctcagcggggcttttggcttcatcatcttagtgtacttgctgatcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 8 - DNA, 3637 bp, Artificial Sequence
  Fusion sequence
atgaccagcagccacgccatgaacatcacccccctggctcagctggccctgctgttctctacactgctgctgcctggcacccaggcactgctggctcctacaacacctgatgccggcagcgccctgaacctgaccttcgacccttggaccagaaccctgacctgggcctgtgataccgccgctggcaacgtgaccgtgaccagctgtaccgtgacctccagagaggccggcatccacagaagagtgtccccattcggctgcagatgctggttcagacggatgatggccctgcaccacggcgtgaccctggacgtgaacggaacagtgggcggagccgccgctcattggagactgagctttgtgaacgagggcgcagctggctctggcgccgagaatctgacctgcgagatcagagccgccagattcctgagctgcgcttggagagagggacctgccgctcctgctgacgtgcggtactctctgagagtgctgaacagcaccggccacgatgtggccagatgcatggctgaccctggcgacgacgtgatcacccagtgtatcgccaacgacctgagcctgctgggcagcgaggcttacctggtcgtgacaggcagatctggcgctggcccagtgcggttcctggatgatgtggtggccacaaaggccctggaaagactgggccctcctagggacgtgaccgccagctgtaacagctcccactgcaccgtgtcttgggcccctccatctacatgggccagcctgacagccagagacttccagttcgaagtgcagtggcagagcgccgagcctggcagcacacctagaaaggtgctggtggtggaagagacaagactggccttccccagccctgctcctcacggcggacacaaagtgaaagtgcgggctggcgacaccagaatgaagcactggggagagtggtcccccgctcaccctctggaagccgaggacacaagagtgccccctagcctgttcgccctggaagctgtgctgatccccgtgggcacaatgggcctgatcatcaccctgatcttcgtgtactgttggctggaaagaatgccccccatcccccctatcaagaacctggaagatctcgtgaccgagtaccagggcaacttctccgcttggagcggcgtgtccaagggcctgacagagagcctgcagcccgactacagcgagagattctgccacgtgtccgagatcccccccaagggcggagcactgggagaaggacctggcggcagtccttgtagcctgcacagcccttactggcccccaccctgctacagcctgaagcctgaggctggttccggagccacgaacttctctctgttaaagcaagcaggagacgtggaagaaaaccccggtcccatggatcagcagatggctctgacatggggcctgtgctacatggccctggtggctctgtgttggggccatggcgtgacagaggccgaggaaaccgtgcccctgaaaaccctgcagtgttacaacgactacaccaatcacatcatctgtagctgggccgacaccgaggatgcccagggactgatcaacatgaccctgtaccaccagctggaaaagaaacagcccgtgtcctgcgagctgagcgaggaactgatgtggagcgagtgccctagcagccacagatgtgtgcctagaagatgcgtgatcccctacaccagattcagcatcaccaacgaggactactacagcttcagacccgacagcgacctgggaatccagctgatggtgcccctggcccagaacgtgcagcctcccctgcctaagaacgtgtccatcagcagcagcgaggaccggttcctgctggaatggagtgtgtctctgggcgacgctcaggtgtcctggctgagcagcaaggacatcgagttcgaggtggcctacaagaggctgcaggacagctgggaggacgcctactctctgcacaccagcaagttccaagtgaacttcgagcccaagctgttcctgcccaacagcatctacgccgccagagtgcggaccagactgagccctggatctagcctgagcggcagacctagcagatggtcccctgaggtgcactgggacagccagcctggcgataaggctcagcctcagaacctgcagtgcttcttcgacggcatccagtccctgcactgcagctgggaagtgtggacccagaccacaggcagcgtgtccttcggcctgttctacagaccaagccccgtggcccccgaagagaagtgtagccctgtcgtgaaagagcccccaggcgccagcgtgtacacaagataccactgcagcctgcccgtgcccgagccttctgctcactctcagtacaccgtgtccgtgaagcacctggaacagggcaagttcatcatgagctacaaccacatccagatggaaccccccacactgaatctgacaaagaacagagacagctactccctgcattgggagacacagaagatggcctacagctttatcgagcacacatttcaggtgcagtacaagaagaagtccgactcttgggaggacagcaagaccgaaaacctggacagagcccacagcatggacctgagtcagctggaacccgacaccagctactgtgcccgcgtcagagtgaagcccatcagcaactacgatggcatctggtctaagtggagcgaagagtacacctggaaaaccgactggatcctgcccatgagctggctgagatacctgctgctggtgctgggctgcttcagcggcttcttcagctgcgtgtacatcctcgtgaagtgcagatacctgggcccctggctgaaaaccgtgctgaagtgccacatccccgaccccagcgagttctttagccagctgtctagccagcacggcggcgacctgcagaagtggctgagttctcctgtgcctctgagcttcttctccccctccggacctgcccctgagatcagcccactggaagtgctggacggcgacagcaaggcagtgcagcttctgctgctgcagaaagacagcgcccctctgcctagccctagcggacatagccaggcctcttgctttaccaaccagggctactttttctttcatctgcccaacgctctggaaatcgagagctgtcaggtgtacttcacctacgacccctgcgtggaagaggaagtggaagaggacggcagcagactgcctgagggctctcctcatcctcctctgctgcctctggctggcgagcaggacgactactgcgctttcccaccaagggacgatctgctgctgtttagccccagcctgagcacccccaacaccgcttacggcggaagcagagcccccgaggaaagaagccctctgtctctgcacgagggcctgccttctctggccagcagagatctgatgggcctgcagcggcctctggaacggatgcctgaaggggatggcgagggactgagcgccaactctagcggagagcaggcctctgtgccagagggcaatctgcacggccaggaccaggatagaggccagggcccaatcctgaccctgaacaccgatgcctatctgagtctgcaggaactgcaggcacaggactccgtgcacctgatctgagacccagctttcttgtacaaagtgg

SEQ ID NO: 9 - protein, 320 aa, Homo sapiens
MLLLVTSLLLCELPHPAFLLIPEKSDLRTVAPASSLNVRFDSRTMNLSWDCQENTTFSKCFLTDKKNRVVEPRLSNNECSCTFREICLHEGVTFEVHVNTSQRGFQQKLLYPNSGREGTAAQNFSCFIYNADLMNCTWARGPTAPRDVQYFLYIRNSKRRREIRCPYYIQDSGTHVGCHLDNLSGLTSRNYFLVNGTSREIGIQFFDSLLDTKKIERFNPPSNVTVRCNTTHCLVRWKQPRTYQKLSYLDFQYQLDVHRKNTQPGTENLLINVSGDLENRYNFPSSEPRAKHSVKIRAADVRILNWSSWSEAIEFGSDDG

SEQ ID NO: 10 - protein, 115 aa, Homo sapiens
PFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 11 - protein, 438 aa, Homo sapiens
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTES

SEQ ID NO: 12 - protein, 311 aa, Homo sapiens
IPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 13 - protein, 22 aa, Artificial Sequence
  Porcine teschovirus-1 2A (P2A) peptide
GSGATNFSLLKQAGDVEENPGP

SEQ ID NO: 14 - protein, 21 aa, Artificial Sequence
  Thoseaasigna virus 2A (T2A) peptide
GSGEGRGSLLTCGDVEENPGP

SEQ ID NO: 15 - protein, 23 aa, Artificial Sequence
  Equine rhinitis A virus (ERAV) 2A (E2A) peptide
GSGQCTNYALLKLAGDVESNPGP

SEQ ID NO: 16 - protein, 25 aa, Artificial Sequence
  Foot-and-Mouth disease virus 2A (F2A) peptide
GSGVKQTLNFDLLKLAGDVESNPGP

SEQ ID NO: 17 - DNA, 66 bp, Artificial Sequence
  Porcine teschovirus-1 2A (P2A) peptide
ggaagcggagctactaacttcagcctgctgaagcaggctggagacgtggaggagaaccctggacct

SEQ ID NO: 18 - DNA, 66 bp, Artificial Sequence
  Porcine teschovirus-1 2A (P2A) peptide - Codon Optimized
ggttccggagccacgaacttctctctgttaaagcaagcaggagacgtggaagaaaaccccggtccc

SEQ ID NO: 19 - DNA, 63 bp, Artificial Sequence
  Thoseaasigna virus 2A (T2A) peptide
ggaagcggagagggcagaggaagtctgctaacatgcggtgacgtcgaggagaatcctggacct

SEQ ID NO: 20 - DNA, 69 bp, Artificial Sequence
  Equine rhinitis A virus (ERAV) 2A (E2A) peptide
ggaagcggacagtgtactaattatgctctcttgaaattggctggagatgttgagagcaaccctggacct

SEQ ID NO: 21 - DNA, 75 bp, Artificial Sequence
  Foot-and-Mouth disease virus 2A (F2A) peptide
ggaagcggagtgaaacagactttgaattttgaccttctcaagttggcgggagacgtggagtccaaccctggacct

SEQ ID NO: 22 - protein, 26 aa, Artificial Sequence
  CSF2RA Transmembrane domain
NLGSVYIYVLLIVGTLVCGIVLGFLF

SEQ ID NO: 23 - protein, 17 aa, Artificial Sequence
  CSF2RB Transmembrane domain
VLALIVIFLTIAVLLAL

SEQ ID NO: 24 - protein, 10 aa, Artificial Sequence
  Gly Ser linker
  VARIANT (2)...(5): any one or all of amino acids 2-5 can either be present or absent.
  VARIANT (7)...(10): any one or all of amino acids 7-10 can either      be present or absent.
GGGGGSSSSS

SEQ ID NO: 25 - protein, 9 aa, Artificial Sequence
  Synthetic Msln406-414 peptide
GQKMNAQAI

(19) United States
(12) Patent Application Publication
(10) Pub. No.: US 2019/0127435 A1
(43) Pub. Date: May 2, 2019

(54) IMMUNOMODULATORY IL2R FUSION PROTEINS AND USES THEREOF

(71) Applicant: FRED HUTCHINSON CANCER RESEARCH CENTER, Seattle, WA (US)

(72) Inventors: Thomas M. SCHMITT, Seattle, WA (US); Philip D. GREENBERG, Mercer Island, WA (US); Ingunn M. STROMNES, Kenmore, WA (US)

(21) Appl. No.: 16/094,420

(22) PCT Filed: Apr. 20, 2017
(86) PCT No.: PCT/US2017/028693

§ 371 Date: Oct. 17, 2018

Related U.S. Application Data
(60) Provisional application No. 62/325,428, filed on Apr. 20, 2016.

(51) Int. Cl. C07K 14/725; C07K 14/715; C07K 14/705; A61K 35/17; C12N 5/0783

(52) U.S. Cl. CPC C07K 14/7051; C07K 14/7155; C07K 14/7153; C07K 14/70521; C07K 2317/622; A61K 35/17; C12N 5/0638; C07K 2319/03; C07K 2319/02; C07K 14/70578

(57) ABSTRACT

The present disclosure relates to fusion proteins containing an extracellular cytokine binding domain and an intracellular signaling domain of one or more IL-2R chains or signaling portion(s) thereof, wherein the cytokine binding domain is not an IL-2 binding domain. The present disclosure also relates to uses of immune cells expressing such fusion proteins to treat certain diseases, such as cancer or infectious disease.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_442WO_SEQUENCE_LISTING.txt. The text file is 48.8 KB, was created on Apr. 20, 2017, and is being submitted electronically via EFS-Web.

### BACKGROUND

T cell-based immunotherapies began to be developed when tumor-reactive T cells were found among a population of tumor-infiltrating lymphocytes (TILs) (Clark et al., Cancer Res. 29:705, 1969). One strategy, known as adoptive T cell transfer, in some contexts involves the isolation of tumor infiltrating lymphocytes pre-selected for tumor-reactivity, clonal expansion of the tumor-reactive T cells induced by anti-CD3 and anti-CD28 antibodies in the presence of IL-2, and finally infusing the expanded cell population back to the tumor-bearing patient (together with chemotherapy and repetitive administration of IL-2) (Dudley et al., Science 298:850, 2002). This form of adoptive T cell therapy with tumor infiltrating lymphocytes can be technically cumbersome and leads to complete remission in only a minor fraction of patients with melanoma and is rarely effective in other cancers (Besser et al., Clin. Cancer Res. 6:2646, 2010).

Isolation of tumor-reactive T cell clones led to the development of another immunotherapeutic approach—the generation of recombinant T cell receptors (TCRs) specific for particular antigens, which may be introduced into T cells, e.g., using a vector delivery system, to confer specificity for a desired target such as a tumor-associated peptide presented by a major histocompatibility complex (MEW) molecule expressed on a tumor cell (known as human leukocyte antigen (HLA) molecule in humans). Another approach introduces a synthetic receptor, termed a chimeric antigen receptor (CAR), which generally contains an antigen-binding domain, which, e.g., in the context of anti-tumor therapy can bind to a tumor-specific or associated antigen, linked to one or more intracellular component comprising an effector domains, such as a primary signaling domain such as a TCR signaling domain or in some contexts costimulatory signaling domains. Unlike administration of TILs, the basic procedure for engineered TCR or CAR T cell immunotherapy is generally to genetically modify human T cells with a transgene encoding a tumor targeting moiety, ex vivo expansion of the recombinant T cells, and transfusing the expanded recombinant T cells back into patients.

Adoptive T cell therapy using T cells expressing recombinant TCRs has been shown to have a promising clinical benefit, especially in certain B cell cancers. However, effective T cell activation often requires or is enhanced by a concurrent co-stimulatory signal (Chen and Flies, Nat. Rev. Immunol. 13: 227-242, 2013). In the tumor microenvironment, co-stimulatory molecules are generally downregulated. As a result, exogenous stimulus via IL-2 is typically needed for T cells that express recombinant TCRs specific for cancer antigens.

Activation of T cells is initiated when the TCR engages a specific peptide presented in MHC on an antigen-presenting cell (APC) (Rossy et al., Frontiers in Immunol. 3:1, 2012). Multiple cytokines, including IL-2, can affect T cell proliferation and survival. The magnitude of the T cell response is regulated in part by signals delivered to T cells through cytokine receptors. The IL-2 receptor (IL-2R) complex is a heterotrimer comprised of a unique a chain (CD25), α β chain shared with the IL-15 receptor, and a _65 chain shared with the IL-4, IL-7, IL-9, and IL-15 receptors, all of which also can deliver proliferative signals (Nelson and Willerford, Adv. Immunol. 70:1, 1998).

CD8⁺ T cells generally lose the ability to produce IL-2 after differentiation into effector T cells (CTLs) (Aruga et al., J. Leukocyte Biol. 61:507, 1997). Differentiated effector CD8⁺ T cells also retain the capacity to secrete many cytokines, including granulocyte-macrophage colony-stimulating factor (GM-CSF) (Aruga et al., 1997), but do not express the GM-CSF receptor. Administration of exogenous IL-2 systemically can be used to prolong CTL longevity in vivo (Cheever and Chen, Immuno. Rev. 157:177, 1997), but IL-2 treatment has been associated with severe toxicity (Dalgleish, Gene Ther. 1:83, 1994).

There remains a need in the immunotherapy field for augmented signaling and effectiveness by means of the IL-2R in responding CD8⁺ T cells in vivo. Presently disclosed embodiments address these needs and provide other related advantages.

### BRIEF DESCRIPTION OF THE DRAWINGS

FIG. 1 shows the relative gene expression of select cytokines and chemokines as determined by quantitative PCR (data represent mean±SEM of 3 independently derived primary invasive tumor (pancreatic ductal adenocarcinoma, PDA) and paired metastatic cell preparations and are normalized to expression in preinvasive cells.

FIG. 2 is an illustration of exemplary fusion proteins of this disclosure. A first fusion protein comprises an extracellular component from CSF2Ra and an intracellular component from IL-2Rγ, and a second fusion protein comprises an extracellular component from CSF2Rβ and an intracellular component from IL-2Rβ. The illustration shows the fusion proteins located in a cell membrane (e.g., T cell membrane) and forming a complex, which is a heterodimer.

FIGS. 3A and 3B show (A) CDR3 sequences of Vα4 and Vβ9 chains cloned from the highest avidity MSLN₄₀₆₋₄₁₄-specific T cell clones isolated from wild-type and Msln^{-/-} mice; and (B) results of flow cytometric assessment of the percentage of donor (gating on Thy1.1+/Vβ9+ (indicative of mesothelin-specific TCR₁₀₄₅⁺)) CD8+ T cells in the blood of animals, following adoptive transfer, that expressed a GM-CSFR extracellular motif (left panel). The detection of the motif on the T cells indicated the expression of the GM-CSF::IL-2R fusion protein. Staining of monocytes (right panel), which express GM-CSFR, served as a positive control.

FIGS. 4A to 4C show (A) the overall percentage of donor (gating on Thy1.1+, CD8+) T cells detected in the blood over time following adoptive transfer (days 0, 5, 10, 15, 20, 25); (B) the percentage of the donor (gating on Thy1.1+/Vβ9+) CD8+ T cells in the blood at day 0 and day 14 (left and right panels, respectively) in which the GM-CSFR extracellular motif was detected, indicating the expression of the GM-CSF::IL-2R fusion protein; (C) shows the relative percentage of donor cells in the blood represented by cells expressing the GM-CSF::IL-2R fusion protein (“GM/IL2R TCR₁₀₄₅”) as compared to those in which the fusion protein was not detected (“TCR₁₀₄₅”), indicating that the fusion protein provided a survival and/or expansion advantage to the mesothelin-targeting TCR₁₀₄₅-expressing T cells in this study.

### DETAILED DESCRIPTION

The instant disclosure provides fusion proteins that modulate signaling in a host cell, such as an immune cell. For example, fusion proteins of this disclosure can provide an activation or proliferation signal to a human T cell, wherein the T cell may optionally be engineered to have a preferred antigen-specific T cell receptor (TCR) or chimeric antigen receptor (CAR) or both. For example, these fusion proteins can interact with a cytokine or chemokine of interest to provide T cells, such as T cells containing an antigen-specific TCR or CAR, a survival and/or expansion advantage, which is consistent with utility of the construct to improve persistence and exposure to transferred cells, including improving efficacy in a tumor microenvironment.

In certain aspects, the present disclosure provides host cells (e.g., immune cells, such as T cells) comprising a fusion protein, vectors encoding fusion proteins, and methods of activating T cells comprising a fusion protein for various therapeutic applications, including the treatment of a disease in subject (e.g., cancer, infectious disease).

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

The term “consisting essentially of” limits the scope of a claim to the specified materials or steps, or to those that do not materially affect the basic characteristics of a claimed invention. For example, a protein domain, region, or module (e.g., a binding domain, hinge region, linker module) or a protein (which may have one or more domains, regions, or modules) “consists essentially of” a particular amino acid sequence when the amino acid sequence of a domain, region, or module or protein includes extensions, deletions, mutations, or any combination thereof (e.g., amino acids at the amino- or carboxy-terminus or between domains) that, in combination, contribute to at most 20% (e.g., at most 15%, 10%, 8%, 6%, 5%, 4%, 3%, 2%, or 1%) of the length of a domain, region, or module or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “heterologous” or “non-endogenous” or “exogenous” refers to any gene, protein, compound, molecule, or activity that is not native to a host cell or a subject, or is any gene, protein, compound, molecule, or activity native to a host or host cell that has been altered or mutated such that the structure, activity or both is different as between the native and mutated molecules. In certain embodiments, heterologous, non-endogenous or exogenous molecules (e.g., receptors, ligands) may not be endogenous to a host cell or subject, but instead nucleic acids encoding such molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a molecule or activity found in or derived from a host cell, species, or strain. For example, a heterologous or exogenous molecule or gene encoding the molecule may be homologous to a native host or host cell molecule or gene that encodes the molecule, respectively, but may have an altered structure, sequence, expression level or combinations thereof. A non-endogenous molecule may be from the same species, a different species, or a combination thereof.

As used herein, the term “endogenous” or “native” refers to a gene, protein, compound, molecule, or activity that is normally present in a host or host cell and has no engineered alterations.

A “binding domain” (also referred to as a “binding region” or “binding moiety”), as used herein, refers to a molecule, such as a peptide, oligopeptide, polypeptide or protein, that possesses the ability to specifically and non-covalently associate, unite, or combine with a target molecule. A binding domain includes any naturally occurring, synthetic, semi-synthetic, or recombinantly produced binding partner for a biological molecule or other target of interest or binding protein thereof. In some embodiments, the binding domain is an antigen-binding domain from, for example, an antibody or T cell receptor (TCR) or comprises a functional binding domain or antigen-binding fragment thereof (e.g., domain antibodies, sFv, scFv, Fab, single chain TCRs (scTCRs), or the like). In other embodiments, a binding domain or binding portions thereof binds to a cytokine or chemokine, such as GM-CSF.

In some embodiments, “specifically binds” refers to an association or union of a binding domain, or a fusion protein thereof, to a target molecule with an affinity or Kₐ (i.e., an equilibrium association constant of a particular binding interaction with units of 1/M) equal to or greater than 10⁵ M⁻¹, or binds to such target molecule while not significantly associating or uniting with any other molecules or components in a sample. Binding domains (or fusion proteins thereof) may be classified as “high affinity” binding domains (or fusion proteins thereof) or “low affinity” binding domains (or fusion proteins thereof). “High affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” binding domains refer to those binding domains with a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). In certain embodiments, a binding domain may have “enhanced affinity,” which refers to a selected or engineered binding domain with stronger binding to a target antigen than a wild type (or parent) binding domain. For example, enhanced affinity may be due to a Ka (equilibrium association constant) for the target antigen that is higher than the wild type binding domain, or due to a K_{d} (dissociation constant) for the target antigen that is less than that of the wild type binding domain, or due to an off-rate (K_{off}) for the target antigen that is less than that of the wild type binding domain. A variety of assays are known for identifying binding domains of the present disclosure that specifically bind a particular target, as well as determining binding domain or fusion protein affinities, such as Western blot, ELISA, and Biacore® analysis (see also, e.g., Scatchard et al., Ann. N.Y. Acad. Sci. 51:660, 1949; and U.S. Pat. Nos. 5,283,173, 5,468,614, or the equivalent).

As used herein, a “fusion protein” refers to a polypeptide that, in a single chain, has at least two distinct domains, wherein the domains are not naturally found together in a protein. A nucleic acid molecule encoding a fusion protein may be constructed using PCR, recombinantly engineered, or the like, or such fusion proteins can be made using methods of protein synthesis. A fusion protein may further contain other components (e.g., covalently bound), such as a tag or bioactive molecule. In certain embodiments, a fusion protein expressed or produced by a host cell (e.g., T cell) locates to the cell surface, where the fusion protein is anchored to the cell membrane with a portion of the fusion protein located extracellularly (e.g., containing a binding domain) and a portion of the fusion protein located intracellularly (e.g., containing a signaling domain).

As used herein, an “extracellular component” refers to a portion or domain of a fusion protein that is located outside of a cell and that is capable of specifically interacting or associating with another molecule or compound to induce a biological effect by transmitting a signal to the intracellular component of the fusion protein. For example, a cytokine binding domain is capable of associating with a specific cytokine and inducing signal transduction into the cell via the intracellular component of the fusion protein.

As used herein, an “intracellular component” refers to a portion or domain of a fusion protein that is located in the cytoplasm of a host cell and that is capable of transmitting signals to the cell via an “intracellular signaling domain” by interacting with a signaling molecule or with another intracellular component.

As used herein, an “intracellular signaling domain” is an intracellular portion of molecule, such as one used in a fusion protein of this disclosure, that can directly or indirectly promote a response, such as a co-stimulatory, positive, or activating biological or physiological response in a cell when receiving the appropriate signal. In certain embodiments, an intracellular signaling domain is part of a protein or protein complex that receives a signal when bound, or itself can bind directly to a target molecule to transmit a signal to other components in the cell. An intracellular signaling domain may directly promote a cellular response when it contains one or more signaling domains or motifs, such as a Box 1 motif (e.g., JAK interacting domain found on a common gamma chain), a kinase domain, an immunoreceptor tyrosine-based activation motif (ITAM), a co-stimulatory domain, or the like. In other embodiments, an intracellular signaling domain will indirectly promote a cellular response by associating with one or more other proteins that in turn directly promote a cellular response.

As used herein, a “portion thereof” refers to a particular region of a protein, such as an extracellular portion, a transmembrane portion or an intracellular portion, or refers to a domain, motif, or fragment of a protein or protein region that retains the function associated with the domain, motif, or fragment of the protein or the protein region. For example, a portion thereof of a cytokine binding domain means a fragment of this domain that is still capable of binding the cytokine.

In certain embodiments, a fusion protein may contain a “linker,” which can provide a spacer function to facilitate the interaction of two single chain fusion proteins, or positioning of one or more binding domains, so that the resulting polypeptide structure maintains a specific binding affinity to a target molecule or maintains signaling activity (e.g., effector domain activity) or both. Exemplary linkers include from one to about ten repeats of GlyₓSer_{y}, wherein x and y are independently an integer from 1 to 5.

“Junction amino acids” or “junction amino acid residues” refer to one or more (e.g., about 2-20) amino acid residues between two adjacent motifs, regions, or domains of a fusion protein, such as between a binding domain and an adjacent hydrophobic component, or on one or both ends of a hydrophobic component. Junction amino acids may result from the construct design of a fusion protein (e.g., amino acid residues resulting from the use of a restriction enzyme site during the construction of a nucleic acid molecule encoding a fusion protein). In certain embodiments, junction amino acids form a linker, such as those having from one to about ten repeats of GlyₓSer_{y}, wherein x and y are independently an integer from 1 to 5.

As used herein, an “immune system cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells). Exemplary immune system cells include a CD4+ T cell, a CD8+ T cell, a CD4-CD8-double negative T cell, a γδ T cell, a regulatory T cell, a natural killer cell, and a dendritic cell. Macrophages and dendritic cells may be referred to as “antigen presenting cells” or “APCs,” which are specialized cells that can activate T cells when a major histocompatibility complex (MEW) receptor on the surface of the APC complexed with a peptide interacts with a TCR on the surface of a T cell.

A “T cell” is an immune system cell that matures in the thymus and produces T cell receptors (TCRs). T cells can be naive (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naive T cells) and effector memory T cells (T_{EM}, decreased expression of CD62L, CCR7, CD28, CD45RA, and increased expression of CD127 as compared to naive T cells or T_{CM}). Effector T cells (T_{E}) refers to antigen-experienced CD8+ cytotoxic T lymphocytes that have decreased expression of CD62L ,CCR7, CD28, and are positive for granzyme and perforin as compared to T_{CM}. Other exemplary T cells include regulatory T cells, such as CD4+ CD25+ (Foxp3+) regulatory T cells and Treg17 cells, as well as Tr1, Th3, CD8+CD28−, and Qa-1 restricted T cells.

“T cell receptor” (TCR) refers to a molecule found on the surface of T cells (or T lymphocytes) that, in association with CD3, is generally responsible for recognizing antigens bound to major histocompatibility complex (MHC) molecules. The TCR has a disulfide-linked heterodimer of the highly variable a and (3 chains (also known as TCRα and TCRβ, respectively) in most T cells. In a small subset of T cells, the TCR is made up of a heterodimer of variable γ and δ chains (also known as TCRγ and TCRδ, respectively). Each chain of the TCR is a member of the immunoglobulin superfamily and possesses one N-terminal immunoglobulin variable domain, one immunoglobulin constant domain, a transmembrane region, and a short cytoplasmic tail at the C-terminal end (see Janeway et al., Immunobiology: The Immune System in Health and Disease, 3^{rd} Ed., Current Biology Publications, p. 4:33, 1997). TCR, as used in the present disclosure, may be from various animal species, including human, mouse, rat, cat, dog, goat, horse, or other mammals. TCRs may be cell-bound (i.e., have a transmembrane region or domain) or in soluble form.

“Major histocompatibility complex molecules” (MHC molecules), which is used interchangeably and is understood to also refer to the human counterpart human leukocyte antigen (HLA molecules), refer to glycoproteins that deliver peptide antigens to a cell surface. MHC class I molecules are heterodimers consisting of a membrane spanning a chain (with three a domains) and a non-covalently associated β2 microglobulin. MHC class II molecules are composed of two transmembrane glycoproteins, α and β, both of which span the membrane. Each chain has two domains. MHC (HLA) class I molecules deliver peptides originating in the cytosol to the cell surface, where peptide:MHC (or peptide:HLA in humans) complex is recognized by CD8⁺ T cells. MHC (HLA) class II molecules deliver peptides originating in the vesicular system to the cell surface, where they are recognized by CD4⁺ T cells. An MHC molecule may be from various animal species, including human, mouse, rat, or other mammals.

“Nucleic acid molecule”, or polynucleotide, may be in the form of RNA or DNA, which includes cDNA, genomic DNA, and synthetic DNA. A nucleic acid molecule may be double stranded or single stranded, and if single stranded, may be the coding strand or non-coding (anti-sense strand). A coding molecule may have a coding sequence identical to a coding sequence known in the art or may have a different coding sequence, which, as the result of the redundancy or degeneracy of the genetic code, or by splicing, can encode the same polypeptide.

Variants of the nucleic acid molecules or polynucleotides of this disclosure are also contemplated. Variant polynucleotides are at least 90%, and preferably 95%, 99%, or 99.9% identical to one of the polynucleotides of defined sequence as described herein, or that hybridizes to one of those polynucleotides of defined sequence under stringent hybridization conditions of 0.015M sodium chloride, 0.0015M sodium citrate at about 65-68° C. or 0.015M sodium chloride, 0.0015M sodium citrate, and 50% formamide at about 42° C. The polynucleotide variants retain the capacity to encode a binding domain or fusion protein thereof having the functionality described herein.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015M sodium chloride, 0.0015M sodium citrate at about 65-68° C. or 0.015M sodium chloride, 0.0015M sodium citrate, and 50% formamide at about 42° C. (see Sambrook et al., Molecular Cloning: A Laboratory Manual, 2nd Ed., Cold Spring Harbor Laboratory, Cold Spring Harbor, N.Y., 1989).

More stringent conditions (such as higher temperature, lower ionic strength, higher formamide, or other denaturing agent) may also be used; however, the rate of hybridization will be affected. In instances wherein hybridization of deoxyoligonucleotides is concerned, additional exemplary stringent hybridization conditions include washing in 6×SSC, 0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides).

A “vector” is a nucleic acid molecule that is capable of transporting another nucleic acid. Vectors may be, for example, plasmids, cosmids, viruses, or phage. An “expression vector” is a vector that is capable of directing the expression of a protein encoded by one or more genes carried by the vector when it is present in the appropriate environment.

“Retroviruses” are viruses having an RNA genome. “Gammaretrovirus” refers to a genus of the retroviridae family. Exemplary gammaretroviruses include mouse stem cell virus, murine leukemia virus, feline leukemia virus, feline sarcoma virus, and avian reticuloendotheliosis viruses.

“Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (Hy); bovine immune deficiency virus (BIV); and simian immunodeficiency virus (SIV).

The terms “identical” or “percent identity,” in the context of two or more polypeptide or nucleic acid molecule sequences, means two or more sequences or subsequences that are the same or have a specified percentage of amino acid residues or nucleotides that are the same over a specified region (e.g., 60%, 65%, 70%, 75%, 80%, 85%, 90%, 91%, 92%, 93%, 94%, 95%, 96%, 97%, 98%, 99%, or 100% identity), when compared and aligned for maximum correspondence over a comparison window, or designated region, as measured using methods known in the art, such as a sequence comparison algorithm, by manual alignment, or by visual inspection. For example, preferred algorithms suitable for determining percent sequence identity and sequence similarity are the BLAST and BLAST 2.0 algorithms, which are described in Altschul et al. (Nucleic Acids Res. 25:3389, 1977) and Altschul et al. (J. Mol. Biol. 215:403, 1990), respectively.

“Treat” or “treatment” or “ameliorate” refers to medical management of a disease, disorder, or condition of a subject (e.g., a human or non-human mammal, such as a primate, horse, dog, mouse, or rat). In general, an appropriate dose or treatment regimen comprising a host cell expressing a fusion protein of this disclosure, and optionally an adjuvant or adjunctive therapy, is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; prolonged survival; or any combination thereof.

A “therapeutically effective amount” or “effective amount” of a fusion protein or cell expressing a fusion protein of this disclosure, in the context of a disease or condition being treated, refers to that amount of fusion protein or number of cells sufficient to result in amelioration of one or more symptoms of the disease being treated in a statistically significant manner (e.g., reducing infection, reducing tumor size, inhibiting cancer growth or the like).

Nucleic Acids, Fusion Proteins, and Host Cells

In certain aspects, the present disclosure provides nucleic acid molecules that encode any one or more of the fusion proteins described herein, which may be fusion proteins comprising an extracellular component comprising all or a portion of a cytokine binding domain, a transmembrane domain and an intracellular component comprising a signaling domain of one or more IL-2R chain or signaling portion(s) thereof, wherein the cytokine binding domain is not an IL-2 binding domain. Such nucleic acid molecules can be inserted into an appropriate vector (e.g., viral vector or non-viral plasmid vector) for introduction in a host cell of interest (e.g., T cell).

In certain embodiments, the present disclosure provides a polynucleotide encoding a fusion protein comprising a transmembrane domain disposed between an extracellular component comprising a cytokine binding domain or portion thereof, and an intracellular component comprising an IL-2R intracellular portion, intracellular signaling domain or a portion thereof. In some embodiments, the encoded intracellular component is comprised of an intracellular portion, intracellular signaling domain or portion thereof from an IL-2Rγ, IL-2Rβ, IL-4R, IL-7R, IL-9R, IL-15R, IL-21R, or any combination thereof. In related embodiments, the encoded intracellular component is comprised of an intracellular portion, intracellular signaling domain or portion thereof from a human IL-2Rγ, human IL-2Rβ, human IL-4R, human IL-7R, human IL-9R, human IL-15R, human IL-21R, or any combination thereof.

In other embodiments, the present disclosure provides a polynucleotide encoding a fusion protein comprising an extracellular component comprising all or a portion of a cytokine binding domain, a transmembrane domain and an intracellular component comprising a signaling domain of one or more IL-2R chain or a signaling portion thereof, wherein the cytokine binding domain is not an IL-2 binding domain. In certain embodiments, a polynucleotide encodes (a) a first fusion protein and all or portion of the cytokine binding domain is a first portion of the cytokine binding domain, and (b) a second fusion protein comprising a second portion of the cytokine binding domain, a second transmembrane domain and a second intracellular component comprising a second IL-2R chain signaling domain or signaling portion thereof, and optionally further comprising a polynucleotide encoding an antigen receptor or portion thereof or an antigen binding protein, such as an antigen-specific TCR or CAR.

In further embodiments, the present disclosure provides a polynucleotide encoding a fusion protein comprising a transmembrane domain disposed between an extracellular component comprising a cytokine binding domain or portion thereof, and an intracellular component comprising an IL-2Rγ intracellular portion, intracellular signaling domain or a portion thereof, optionally a human IL-2Rγ. In particular embodiments, a polynucleotide encodes a fusion protein comprising a transmembrane domain disposed between an extracellular component comprising a cytokine binding domain or portion thereof, and an intracellular component, wherein a non-cytokine binding portion of the extracellular component, the transmembrane domain, and the intracellular component of the encoded fusion protein has at least 85%, at least 90%, at least 95%, at least 96%, at least 97%, at least 98%, or at least 99% amino acid sequence identity to the amino acid sequence set forth in SEQ ID NO.:10. In still further embodiments, the encoded non-cytokine binding extracellular portion, transmembrane domain, and intracellular portion of the encoded fusion protein is comprised of or consists of the amino acid sequence set forth in SEQ ID NO.:10.

In certain embodiments, any of the aforementioned polynucleotides encode a fusion protein comprising an extracellular component comprising a cytokine binding domain and a portion of an IL-2R chain comprising a non-cytokine binding extracellular portion, a transmembrane domain of an IL-2R chain, and an intracellular portion of an IL-2R chain, such as an IL-2Rγ or human IL-2Rγ. In further embodiments, the non-cytokine binding extracellular portion, transmembrane domain, and intracellular portion of the IL-2R chain is encoded by a polynucleotide having at least 80%, at least 85%, at least 90%, at least 95%, or at least 98% identity to nucleotides 961-1,308 of SEQ ID NO.:6. In still further embodiments, the non-cytokine binding extracellular portion, transmembrane domain, and intracellular portion of the IL-2R chain is encoded by a polynucleotide comprising or consisting of nucleotides 961-1,308 of SEQ ID NO.:6.

In yet further embodiments, the present disclosure provides a polynucleotide encoding a fusion protein comprising a transmembrane domain disposed between an extracellular component comprising a cytokine binding domain or portion thereof, and an intracellular component comprising an IL-2Rβ intracellular portion, intracellular signaling domain or a portion thereof, optionally a human IL-2Rβ. In particular embodiments, a polynucleotide encodes a fusion protein comprising a transmembrane domain disposed between an extracellular component comprising a cytokine binding domain or portion thereof, and an intracellular component, wherein the transmembrane domain and the intracellular component of the encoded fusion protein has at least 85%, at least 90%, at least 95%, at least 96%, at least 97%, at least 98%, or at least 99% amino acid sequence identity to the sequence set forth in SEQ ID NO.:12. In still further embodiments, the encoded transmembrane domain and intracellular portion of the encoded fusion protein is comprised of or consists of the amino acid sequence set forth in SEQ ID NO.:12.

In certain embodiments, any of the aforementioned polynucleotides encode a fusion protein comprising an extracellular component comprising a cytokine binding domain, a transmembrane domain of an IL-2R chain, and an intracellular portion of an IL-2R chain, such as an IL-2Rβ or human IL-2Rβ. In further embodiments, the transmembrane domain and intracellular portion of the IL-2R chain is encoded by a polynucleotide having at least 80%, at least 85%, at least 90%, at least 95%, or at least 98% identity to nucleotides 1,315-2,250 of SEQ ID NO.:7. In still further embodiments, the non-cytokine binding extracellular portion, transmembrane domain, and intracellular portion of the IL-2R chain is encoded by a polynucleotide comprising or consisting of nucleotides 1,315-2,250 of SEQ ID NO.:7.

In any of the aforementioned embodiments, the encoded extracellular component comprises an extracellular domain or portion thereof of a CSF2RA (also referred to as GM-CSFR or CSF2R), CSF2RB (also referred to as IL3RB, IL5RB, CD131), CSF1R (also referred to as M-CSFR or CSFR), CSF3R (also referred to as G-CSFR or CD114), CXCR2 (also referred to as IL8RA, IL8RB, IL8R2 or CD182), or CCR8 (also referred to as CY6 or TER1). In further embodiments, the encoded extracellular component comprises an extracellular domain or portion thereof of a human CSF2RA, human CSF2RB, human CSF1R, human CSF3R, human CXCR2, or human CCR8.

In any of the aforementioned embodiments, the encoded cytokine binding domain or binding portion thereof specifically binds to a GM-CSF (also referred to as CSF2), M-CSF (also referred to as CSF1), G-CSF (also referred to as CSF3), CXCL1, CXCL2, or CCL1. In further embodiments, the encoded cytokine binding domain or binding portion thereof specifically binds to a human GM-CSF, a human M-CSF, a human G-CSF, a human CXCL1, a human CXCL2, or a human CCL1.

In certain embodiments, any of the aforementioned polynucleotides encode a fusion protein comprising a cytokine binding domain specific for CSF2, such as an extracellular portion of a CSF2RA or CSF2RB. In further embodiments, the extracellular portion of a CSF2RA is encoded by a polynucleotide having at least 80%, at least 85%, at least 90%, at least 95%, or at least 98% identity to nucleotides 1-960 of SEQ ID NO.:6. In still further embodiments, the extracellular portion of a CSF2RA is encoded by a polynucleotide comprising or consisting of nucleotides 1-960 of SEQ ID NO.:6. In related embodiments, the encoded extracellular portion of a CSF2RA is at least 85%, at least 90%, at least 95%, at least 96%, at least 97%, at least 98%, or at least 99% identical to the amino acid sequence set forth in SEQ ID NO.:9. In still further embodiments, the encoded extracellular portion of a CSF2RA is comprised of or consists of the amino acid sequence set forth in SEQ ID NO.:9. In other embodiments, the extracellular portion of a CSF2RB is encoded by a polynucleotide having at least 80%, at least 85%, at least 90%, at least 95%, or at least 98% identity to nucleotides 1-1,314 of SEQ ID NO.:7. In still further embodiments, the extracellular portion of a CSF2RB is encoded by a polynucleotide comprising or consisting of nucleotides 1-1,314 of SEQ ID NO.:6. In related embodiments, the encoded extracellular portion of a CSF2RB is at least 85%, at least 90%, at least 95%, at least 96%, at least 97%, at least 98%, or at least 99% identical to the amino acid sequence set forth in SEQ ID NO.:11. In yet other embodiments, the encoded extracellular portion of a CSF2RB is comprised of or consists of the amino acid sequence set forth in SEQ ID NO.:11.

In certain embodiments, any of the aforementioned polynucleotides encode a fusion protein comprising a transmembrane domain, such as a transmembrane domain of an IL-2RG, IL-2RB, CSF2RA, CSF2RB, CSF1R, CSF3R, CXCR2, CCR8, IL-2RA, IL-4R, IL-7R, IL-9R, IL-15R, IL-21R, CD2, CD3ε, CD3δ, CD3ζ, CD25, CD27, CD28, CD40, CD79A, CD79B, CD80, CD86, CD95 (Fas), CD134 (OX40), CD137 (4-1BB), CD150 (SLAMF1), CD152 (CTLA4), CD200R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GAL9, KIR, Lck, LAT, LRP, NKG2D, NOTCH1, NOTCH2, NOTCH3, NOTCH4, PTCH2, ROR2, Ryk, Slp76, SIRPα, pTα, TCRα, TCRβ, TIM3, TRIM, LPA5, or Zap70. In some embodiments, the encoded transmembrane domain is of a human IL-2RG, a human IL-2RB, a human CSF2RA, a human CSF2RB, a human CSF1R, a human CSF3R, a human CXCR2, a human CCR8, a human IL-2RA, a human IL-4R, a human IL-7R, a human IL-9R, a human IL-15R, a human IL-21R, a human CD2, a human CD3ε, a human CD3δ, a human CD3ζ, a human CD25, a human CD27, a human CD28, a human CD40, a human CD79A, a human CD79B, a human CD80, a human CD86, a human CD95 (Fas), a human CD134 (OX40), a human CD137 (4-1BB), a human CD150 (SLAMF1), a human CD152 (CTLA4), a human CD200R, a human CD223 (LAG3), a human CD270 (HVEM), a human CD272 (BTLA), a human CD273 (PD-L2), a human CD274 (PD-L1), a human CD278 (ICOS), a human CD279 (PD-1), a human CD300, a human CD357 (GITR), a human A2aR, a human DAP10, a human FcRα, a human FcRβ, a human FcRγ, a human Fyn, a human GAL9, a human KIR, a human Lck, a human LAT, a human LRP, a human NKG2D, a human NOTCH1, a human NOTCH2, a human NOTCH3, a human NOTCH4, a human PTCH2, a human ROR1, a human ROR2, a human Ryk, a human Slp76, a human SIRPα, a human pTα, a human TCRα, a human TCRβ, a human TIM3, a human TRIM, a human LPA5, or a human Zap70. In particular embodiments, any of the aforementioned polynucleotides encode a fusion protein comprising a transmembrane domain that is at least 90%, at least 95%, at least 96%, at least 97%, at least 98%, or at least 99% identical to the amino acid sequence set forth in SEQ ID NO.:22 or 23. In other embodiments, the encoded transmembrane domain comprises or consists of the amino acid sequence set forth in SEQ ID NO.:22 or 23.

In the case of a polynucleotide encoding an antigen binding protein, such an encoded antigen binding protein may comprise a transmembrane domain according to any of the aforementioned transmembrane domain embodiments.

In certain embodiments, the present disclosure provides a polynucleotide encoding a fusion protein comprising a transmembrane domain disposed between an extracellular component comprising a cytokine binding domain or portion thereof, and an intracellular component; wherein the encoded extracellular component comprises an extracellular portion of a CSF2RA and a non-cytokine binding extracellular portion of IL-2Rγ, and the encoded transmembrane domain and encoded intracellular component comprise a portion of IL-2Rγ. In further embodiments, a polynucleotide encodes a fusion protein having at least 90%, at least 95%, at least 96%, at least 97%, at least 98%, or at least 99% identity with the amino acid sequence as set forth in SEQ ID NO.:1. In still further embodiments, a polynucleotide encodes a fusion protein comprising or consisting of the amino acid sequence as set forth in SEQ ID NO.:1.

In certain embodiments, the present disclosure provides a polynucleotide encoding a fusion protein comprising a transmembrane domain disposed between an extracellular component comprising a cytokine binding domain or portion thereof, and an intracellular component; wherein the encoded extracellular component comprises an extracellular portion of a CSF2RB, and the encoded transmembrane domain and encoded intracellular component comprise a portion of IL-2Rβ. In further embodiments, a polynucleotide encodes a fusion protein having at least 90%, at least 95%, at least 96%, at least 97%, at least 98%, or at least 99% identity with the amino acid sequence as set forth in SEQ ID NO.:2. In further embodiments, a polynucleotide encodes a fusion protein comprising or consisting of the amino acid sequence as set forth in SEQ ID NO.:2.

The first 22 amino acids of SEQ ID NOS.:1 and 9, and the first 16 amino acids of SEQ ID NOS.:2 and 11, correspond to a signal sequence for human CSF2RA and CSF2RB, respectively. In certain embodiments wherein a fusion protein of SEQ ID NOS.:1, 2, 9 and 11 is expressed on the surface of a host cell will be a mature protein—that is, the mature protein lacks the signal sequence. For example, a mature protein of CSF2RA according to SEQ ID NO.: 9 corresponds to amino acids 23-320 of SEQ ID NO.:9, and a mature protein of CSF2RB according to SEQ ID NO.:11 corresponds to amino acids 17-438 of SEQ ID NO.:11.

In some embodiments, a polynucleotide encodes a fusion protein comprising an intracellular signaling domain or functional fragment or portion thereof from IL-2R, such as IL-2Rγ or IL-2Rβ. In other embodiments, a polynucleotide encodes an antigen binding protein coprising an intracellular signaling domain or functional fragment or portion thereof from a CD3ε, CD3δ, CD3ζ, CD25, CD27, CD28, CD40, CD47, CD79A, CD79B, CD134 (OX40), CD137 (4 1BB), CD150 (SLAMF1), CD278 (ICOS), CD357 (GITR), CARD11, DAP10, DAP12, FcRα, FcRβ, FcRγ, Fyn, Lck, LAT, LRP, NKG2D, NOTCH1, NOTCH2, NOTCH3, NOTCH4, ROR2, Ryk, Slp76, pTα, TCRα, TCRβ, TRIM, Zap70, PTCH2, or any combination thereof. In some embodiments, an encoded intracellular signaling domain or functional fragment or portion thereof of a fusion protein or antigen binding protein does not comprise a CD3ζ.

As used herein, the term “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering. Genetic alterations include, for example, modifications introducing expressible nucleic acid molecules encoding proteins, fusion proteins or enzymes, or other nucleic acid molecule additions, deletions, substitutions or other functional disruption of a cell's genetic material. Additional modifications include, for example, non-coding regulatory regions in which the modifications alter expression of a gene or operon. In certain embodiments, a cell, such as a T cell, obtained from a subject may be converted into a non-natural or recombinant cell (e.g., a non-natural or recombinant T cell) by introducing a nucleic acid that encodes a fusion protein as described herein and whereby the cell expresses a fusion protein.

In certain embodiments, a polynucleotide encodes a plurality of fusion proteins, a plurality of antigen binding proteins, or a combination thereof, wherein two or more of the plurality of fusion proteins, antigen binding proteins, or combinations thereof are separated by a cleavage site. In some embodiments, a cleavage site comprises a protease cleavage site of 2 to about 20 amino acids amino-terminal to a fusion protein or antigen binding protein, a protease cleavage site of 2 to about 20 amino acids carboxy-terminal to a fusion protein or antigen binding protein, a self-cleaving amino acid sequence, or a combination thereof.

In certain embodiments, an encoded cleavage site is a self-cleaving amino acid sequence comprising a 2A peptide from porcine teschovirus-1 (P2A) (SEQ ID NO.:13), Thosea asigna virus (T2A) (SEQ ID NO.:14), equine rhinitis A virus (E2A) (SEQ ID NO.:15), foot-and-mouth disease virus (F2A) (SEQ ID NO.:16), or any combination thereof (see, e.g., Kim et al., PLOS One 6:e18556, 2011). In further embodiments, a polynucleotide that encodes a plurality of fusion proteins, a plurality of antigen binding proteins, or a combination thereof includes a sequence encoding a self-cleaving peptide located between two or more of the proteins, which may be: (a) a P2A peptide encoded by a polynucleotide as set forth in SEQ ID NO.:17; (b) a P2A peptide encoded by a codon optimized polynucleotide as set forth in SEQ ID NO.:18; (c) a T2A peptide is encoded by a polynucleotide as set forth in SEQ ID NO.:19; (d) an E2A peptide is encoded by a polynucleotide as set forth in SEQ ID NO.:20; (e) a F2A peptide is encoded by a polynucleotide as set forth in SEQ ID NO.:21; or (f) any combination thereof.

In certain embodiments, a polynucleotide of this disclosure a first fusion protein, a second fusion protein and optionally an antigen binding protein, wherein the first fusion protein comprises a transmembrane domain disposed between an extracellular component comprising a cytokine binding domain or portion thereof, and an intracellular component that comprises,consists of, or has at least 90% identity to, an IL-2Rγ, optionally a human IL-2Rγ, intracellular portion or intracellular signaling domain or portion thereof, or optionally has at least 85%, at least 90%, at least 95%, at least 96%, at least 97%, at least 98%, or at least 99% amino acid sequence identity to the amino acid sequence set forth in SEQ ID NO.:10. In some embodiments, a polynucleotide encodes a first fusion protein having at least 90%, at least 95%, at least 96%, at least 97%, at least 98%, or at least 99% identity with the amino acid sequence as set forth in SEQ ID NO.:1. In still further embodiments, a polynucleotide encodes a first fusion protein comprising or consisting of the amino acid sequence as set forth in SEQ ID NO.:1.

In further embodiments, a polynucleotide of this disclosure encodes a first fusion protein, a second fusion protein and optionally an antigen binding protein, wherein the encoded first fusion protein comprises a transmembrane domain disposed between an extracellular component comprising a cytokine binding domain or portion thereof, and an intracellular component that comprises,consists of, or has at least 90% identity to, an IL-2Rβ, optionally a human IL-2Rβ, intracellular portion or intracellular signaling domain or portion thereof, or optionally has at least 85%, at least 90%, at least 95%, at least 96%, at least 97%, at least 98%, or at least 99% amino acid sequence identity to the amino acid sequence set forth in SEQ ID NO.:12. In some embodiments, a polynucleotide encodes a first fusion protein having at least 90%, at least 95%, at least 96%, at least 97%, at least 98%, or at least 99% identity with the amino acid sequence as set forth in SEQ ID NO.:2. In further embodiments, a polynucleotide encodes a first fusion protein comprising or consisting of the amino acid sequence as set forth in SEQ ID NO.:2.

In still further embodiments, a polynucleotide of this disclosure encodes a first fusion protein, a second fusion protein, and optionally an antigen binding protein, wherein the encoded first fusion protein has at least 85%, at least 90%, at least 95%, at least 96%, at least 97%, at least 98%, or at least 99% amino acid sequence identity to the amino acid sequence set forth in SEQ ID NO.:1 or set forth in amino acids 23-435 (mature fusion protein) of SEQ ID NO.:1; the encoded second fusion protein has at least 90%, at least 95%, at least 96%, at least 97%, at least 98%, or at least 99% amino acid sequence identity to the amino acid sequence set forth in SEQ ID NO.:2 or set forth in amino acids 17-749 (mature fusion protein) of SEQ ID NO.:2; and the optional antigen binding protein comprises an antigen-specific TCR or an antigen-specific CAR, wherein the antigen is optionally a cancer-specific antigen, such as a WT-1, mesothelin, ROR1 or cyclin-A1 antigen.

In each of these embodiments, a first fusion protein, a second fusion protein, and optionally an antigen binding protein may be all encoded by a single polynucleotide, or they all may be encoded by two polynucleotides (e.g., a first polynucleotide encodes the first fusion protein and a second polynucleotide encodes the second fusion protein and the optional antigen binding protein; or a first polynucleotide encodes the second fusion protein and a second polynucleotide encodes the first fusion protein and the optional antigen binding protein; or a first polynucleotide encodes the first fusion protein and second fusion protein, and a second polynucleotide encodes the optional antigen binding protein, or any combination thereof).

In certain embodiments, a single polynucleotide encodes a first fusion protein of this disclosure and a second fusion protein of this disclosure, wherein a polynucleotide encoding a a self-cleaving peptide as set forth in any one of SEQ ID NOS.:17-21 is disposed between and links the polynucleotide encoding the first fusion protein with the polynucleotide encoding the second fusion protein. In particular embodiments, the first fusion protein is encoded by a polynucleotide having at least 80%, at least 85%, at least 90%, at least 95%, or at least 98% identity to the nucleotide sequence set forth in SEQ ID NO.:6, and the second fusion protein is encoded by a polynucleotide having at least 80%, at least 85%, at least 90%, at least 95%, or at least 98% identity to the nucleotide sequence set forth in SEQ ID NO.:7. In further embodiments, the first fusion protein is encoded by a polynucleotide comprising or consisting of the nucleotide sequence set forth in SEQ ID NO.:6, and the second fusion protein is encoded by a polynucleotide comprising or consisting of the nucleotide sequence set forth in SEQ ID NO.:7.

In any of the aforementioned embodiments, a polynucleotide encoding a fusion protein, an antigen binding protein or both, of this disclosure, may be codon optimized to enhance or maximize expression in certain types of cells, such as T cells (Scholten et al., Clin. Immunol. 119: 135-145, 2006).

Any of the polynucleotides of this disclosure may be contained in a vector or delivered to a host cell (e.g., T cell) via a vector. A vector that encodes a core virus is referred to herein as a “viral vector.” There are a large number of available viral vectors suitable for use with the compositions of the instant disclosure, including those identified for human gene therapy applications (see Pfeifer and Verma, Ann. Rev. Genomics Hum. Genet. 2:177, 2001). Suitable viral vectors include vectors based on RNA viruses, such as retrovirus-derived vectors, e.g., Moloney murine leukemia virus (MLV)-derived vectors, and include more complex retrovirus-derived vectors, e.g., lentivirus-derived vectors. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing chimeric antigen receptor transgenes are known in the art and have been previous described, for example, in U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; Verhoeyen et al., Methods Mol. Biol. 506:97, 2009. Retroviral and lentiviral vector constructs and expression systems are also commercially available.

In certain embodiments, a viral vector is used to introduce a non-endogenous polynucleotide encoding a fusion protein as disclosed herein or a non-endogenous polynucleotide encoding an antigen binding protein specific for a target as disclosed herein, or both. A viral vector may be a retroviral vector or a lentiviral vector. A viral vector may also include nucleic acid sequences encoding a marker for transduction. Transduction markers for viral vectors are known in the art and include selection markers, which may confer drug resistance, or detectable markers, such as fluorescent markers or cell surface proteins that can be detected by methods such as flow cytometry. In particular embodiments, a viral vector further comprises a gene marker for transduction comprising green fluorescent protein (GFP), an extracellular domain of human CD2, or a truncated human EGFR (huEGFRt; see Wang et al., Blood 118:1255, 2011). When a viral vector genome comprises a plurality of nucleic acid sequences to be expressed in a host cell as separate transcripts, the viral vector may also comprise additional sequences between the two (or more) transcripts allowing bicistronic or multicistronic expression. Examples of such sequences used in viral vectors include internal ribosome entry sites (IRES), furin cleavage sites, viral 2A peptide, or any combination thereof.

Other vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5: 1517, 1998).

Other vectors recently developed for gene therapy uses can also be used with the compositions and methods of this disclosure. Such vectors include those derived from baculoviruses and a-viruses (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors). In some embodiments, a viral or plasmid vector further comprises a gene marker for transduction (e.g. green fluorescent protein, huEGFRt).

In some embodiments, a vector comprises a polynucleotide as disclosed herein that encodes more than one fusion protein, and optionally containing a polynucleotide that encodes an antigen-binding protein of this disclosure. For example, a vector may contain a polynucleotide that encodes two different fusion proteins, and optionally containing a polynucleotide that encodes an antigen-binding protein of this disclosure.

In some embodiments, a vector comprising a polynucleotide encoding a fusion protein as disclosed herein may further encode, or comprise a polynucleotide that encodes, an antigen-specific TCR or CAR. In some embodiments, the antigen-specific TCR is exogenous. In some embodiments, the antigen-specific TCR is specific to a HLA (MHC) class I restricted antigen. In some embodiments, the antigen is a cancer-specific antigen. Embodiments wherein the cancer-specific antigen comprises WT-1, mesothelin, ROR1 or cyclin-A1 are also within the scope of this disclosure.

Any of the polynucleotides disclosed herein may be contained in a host cell, wherein the host cell expresses and produces a fusion protein, an antigen binding protein, or both. In certain aspects, the present disclosure provides a host cell comprising a fusion protein and an antigen binding protein, wherein the fusion protein comprises a transmembrane domain disposed between an extracellular component comprising a cytokine binding domain or portion thereof, and an intracellular component comprising an IL-2R intracellular portion, intracellular signaling domain or portions thereof; and wherein the antigen binding protein is a T cell receptor (TCR); a chimeric antigen receptor (CAR); or optionally a plurality of antigen binding proteins. In some embodiments, a host cell of this disclosure comprises a plurality of antigen binding proteins, such as both a TCR and a CAR.

In further aspects, the present disclosure provides a host cell comprising a first fusion protein, a second fusion protein and optionally an antigen binding protein, wherein the first fusion protein comprises a transmembrane domain disposed between an extracellular component comprising a cytokine binding domain or portion thereof, and an intracellular component that is comprised of, or has at least 90% identity to, an IL-2Rγ, optionally human IL-2Rγ, intracellular portion or intracellular signaling domain or portion thereof, or optionally has at least 85%, at least 90%, at least 95%, at least 96%, at least 97%, at least 98%, or at least 99% amino acid sequence identity to the amino acid sequence set forth in SEQ ID NO.:10; wherein the second fusion protein comprises a transmembrane domain disposed between an extracellular component comprising a cytokine binding domain, or portion thereof, and an intracellular component is comprised of, or has at least 90% identity to, an IL-2R β, optionally human IL-2Rβ, intracellular signaling domain or portion thereof and/or a signaling domain or portion thereof of an IL-4R, IL-7R, IL-9R, IL-15R or IL-21R chain, or optionally has at least 85%, at least 90%, at least 95%, at least 96%, at least 97%, at least 98%, or at least 99% amino acid sequence identity to the amino acid sequence set forth in SEQ ID NO.:12; and wherein the optional antigen binding protein comprises a T cell receptor (TCR); a chimeric antigen receptor (CAR); and optionally comprises a plurality of antigen binding proteins. In some embodiments, a host cell of this disclosure comprises a plurality of antigen binding proteins, such as both a TCR and a CAR.

In further aspects, the present disclosure provides a host cell comprising a polynucleotide that encodes a first fusion protein, a second fusion protein, and optionally an antigen binding protein, wherein the encoded first fusion protein has at least 85%, at least 90%, at least 95%, at least 96%, at least 97%, at least 98%, or at least 99% amino acid sequence identity to the amino acid sequence set forth in amino acids 23-435 (mature fusion protein) of SEQ ID NO.:1; the encoded second fusion protein has at least 90%, at least 95%, at least 96%, at least 97%, at least 98%, or at least 99% amino acid sequence identity to the amino acid sequence set forth in amino acids 17-749 (mature fusion protein) of SEQ ID NO.:2; and the optional antigen binding protein comprises an antigen-specific TCR or an antigen-specific CAR, wherein the antigen is optionally a cancer-specific antigen, such as a WT-1, mesothelin, ROR1 or cyclin-A1 antigen.

In certain embodiments, a host cell containing a polynucleotide of this disclosure comprises at least two encoded fusion proteins that are capable of associating to form a heteromultimer on the host cell surface, optionally wheren the heteromultimer on the host cell surface is a heterodimer or heterotrimer.

In further embodiments, the fusion proteins each comprise a different extracellular component, wherein the different extracellular components are capable of associating with each other to form a functional cytokine binding domain. In particular embodiments, one of the different encoded extracellular components is comprised of, or has at least 90% amino acid identity to the amino acid sequence of, a CSF2Rα, optionally a human CSF2Rα, extracellular portion, extracellular cytokine binding domain or portion thereof, or optionally has at least 90% amino acid identity to the amino acid sequence of SEQ ID NO.:9, and the other different extracellular component is comprised of, or has at least 90% amino acid identity to the amino acid sequence of, a CSF2Rβ, optionally human CSF2Rβ, extracellular portion, extracellular cytokine binding domain or portion thereof, or optionally has at least 90% amino acid identity to the amino acid sequence of SEQ ID NO.:11. In any of these embodiments, the fusion proteins each comprise a different intracellular component, wherein the different intracellular components are capable of associating with each other to form a functional intracellular signaling domain. In further embodiments, at least one of the different intracellular components is comprised of, or has at least 90% identity to, an IL-2Rγ intracellular portion, intracellular signaling domain or portions thereof, or optionally has at least 90% amino acid sequence identity to the amino acid sequence of SEQ ID NO.:10. In yet further embodiments, at least one of the different intracellular components is comprised of, or has at least 90% identity to, an IL-2Rβ, IL-4RA, IL-7R, IL-15RA, or IL-21R intracellular portion, intracellular signaling domain or portions thereof, which in each case, individually, is optionally human-derived, or optionally has at least 90% identity to SEQ ID NO.:12.

In other embodiments, a host cell containing a polynucleotide that encodes a fusion protein or a plurality of fusion proteins of this disclosure, may further contain a polynucleotide that encodes an antigen binding protein. In some embodiments, the encoded antigen binding protein is a T cell receptor (TCR), or optionally is an antigen-specific TCR, optionally the TCR binds to an antigen::HLA complex with high affinity, such as at a Kₐ equal to or greater than 10⁷M⁻¹. In further embodiments, the encoded antigen-specific TCR is heterologous to the host cell, or to a subject to whom the host cell will be administered. In particular embodiments, the encoded TCR is specific to a HLA class I restricted antigen. In any of the aforementioned embodiments, the antigen binding protein is specific for a cancer-specific antigen, such as WT-1, mesothelin, ROR1 or cyclin-A1. In some embodiments, the TCR is WT-1 specific TCR designated as C4.

In still further embodiments, a host cell containing a polynucleotide that encodes a fusion protein or a plurality of fusion proteins of this disclosure, may further contain a polynucleotide that encodes an antigen binding protein, wherein the antigen binding protein is a CAR. Exemplary CARs expressed the host cell may comprise an extracellular antigen binding domain and an intracellular signaling domain capable of delivering a primary signal to a T cell and optionally a costimulatory domain; or the intracellular signaling domain comprises an intracellular signaling domain of a costimulatory molecule, such as from CD28, CD137 (4-1BB), or ICOS. In some embodiments, the encoded intracellular signaling domain comprises an intracellular signaling domain of a CD3ε, CD3δ, CD3ζ, CD25, CD27, CD28, CD40, CD47, CD79A, CD79B, CD134 (OX40), CD137 (4-1BB), CD150 (SLAMF1), CD278 (ICOS), CD357 (GITR), CARD11, DAP10, DAP12, FcRα, FcRβ, FcRγ, Fyn, Lck, LAT, LRP, NKG2D, NOTCH1, NOTCH2, NOTCH3, NOTCH4, ROR2, Ryk, Slp76, pTα, TCRα, TCRβ, TRIM, Zap70, PTCH2, or any combination thereof and/or wherein the intracellular signaling portion of the chimeric antigen receptor comprises a primary activation signaling domain, which optionally is derived from CD3 ζ, and does not comprise a costimulatory domain and/or does not comprise a CD28 signaling domain, a 4-1BB signaling domain and/or an ICOS signaling domain.

In certain embodiments, the encoded intracellular signaling domain comprises a costimulatory domain of: (a) a CD137 (4-1BB), CD27, CD28, ICOS, OX40 (CD134), or any combination thereof; (b) a CD137 (4-1BB) or CD28, or any combination thereof; (c) a CD28; or (d) a CD137 (4-1BB). In particular embodiments, the encoded intracellular signaling domain comprises a second intracellular signaling domain, such as an intracellular signaling domain of a CD137 (4-1BB).

In further embodiments, the encoded antigen binding domain of the CAR comprises an antibody binding fragment or scFv specific for the antigen. In some embodiments, a host cell as described herein comprises at least two antigen binding proteins, wherein the at least two antigen binding proteins include a TCR and a CAR. In certain embodiments, the expression of the fusion protein in a T cell comprising a TCR or chimeric antigen receptor specific for an antigen results in at least about a 1.5-fold, 2-fold, or 3-fold increase in survival, expansion, cytotoxicity, cytokine secretion, and/or response to multiple rounds of stimulation, by the T cell, in response to binding of the antigen and/or following administration to a subject, and/or results in at least about a 1.5-fold, 2-fold, or 3-fold increase in time of survival, disease-free survival, or amelioration of one or more disease symptom, of a subject to which the cell is administered, as compared to a cell substantially the same as the T cell but not containing the fusion protein.

Exemplary host cells for use with the fusion proteins, antigen binding proteins and polynucleotides encoding the same of this disclosure includes an immune system cell, such as a T cell. A T cell may be a CD4+ T cell or a CD8+ T cell.

In some embodiments, host cells capable of expressing a fusion protein of this disclosure on the cell surface are immune cells. In some embodiments, host cells capable of expressing a fusion protein of this disclosure on the cell surface are T cells, including primary cells or cell lines derived from human, mouse, rat, or other mammals. If obtained from a mammal, a T cell can be obtained from numerous sources, including blood, bone marrow, lymph node, thymus, or other tissues or fluids. A T cell may be enriched or purified. T cell lines are well known in the art, some of which are described in Sandberg et al., Leukemia 21:230, 2000. In certain embodiments, T cells that lack endogenous expression of TCRα and β chains are used. Such T cells may naturally lack endogenous expression of TCRα and β chains or may have been modified to block expression (e.g., T cells from a transgenic mouse that does not express TCR a and β chains or cells that have been manipulated to inhibit expression of TCR α and β chains) or to knockout TCRα chain, TCRβ chain, or both genes. In some embodiments, T cells may be engineered to express a TCR specific to a particular antigen.

In certain embodiments, a host cell transfected to express a fusion protein of this disclosure is a functional T cell, such as a virus-specific T cell, a tumor antigen specific cytotoxic T cell, a naive T cell, a memory stem T cell, a central or effector memory T cell, γδ T cells, or a CD4+ CD25+ regulatory T cell. In further embodiments, a nucleic acid molecule encoding a fusion protein of this disclosure is introduced into bulk CD8+ T cells, naive CD8+ T cells, CD8+ T_{CM} cells, CD8+ T_{EM} cells, or any combination thereof. In still further embodiments, a nucleic acid molecule encoding a fusion protein of this disclosure is introduced into bulk CD4+ T cells, naive CD4+ T cells, CD4+ T_{CM} cells, CD4+ T_{EM} cells, or any combination thereof. In other embodiments, a nucleic acid molecule encoding a fusion protein of this disclosure is introduced into a population of T cells enriched for naive CD8+ T cells and CD8+ T_{CM} cells. In still other embodiments, a nucleic acid molecule encoding a fusion protein of this disclosure is introduced into a population of T cells enriched for naive CD4+ T cells and CD4+ T_{CM} cells. In any of the aforementioned embodiments, the T cells further contain a nucleic acid molecule encoding an engineered antigen-specific T cell receptor (TCR), an engineered antigen-specific high affinity TCR, an exogenous co-stimulatory molecule, a chimeric antigen receptor (CAR), or any combination thereof.

In certain embodiments, a host cell transfected to express a fusion protein of this disclosure is a functional natural killer cell.

One or more growth factor cytokines that promote proliferation of T cells expressing a fusion protein of this disclosure may be added to the culture used to expand T cells. The cytokines may be human or non-human. Exemplary growth factor cytokines that may be used promote T cell proliferation include GM-CSF, IL-2, IL-15, or the like.

In certain embodiments, a host T cell transfected to express a fusion protein of this disclosure is a CD4⁻ T cell that also expresses an antigen-specific high-affinity TCR specific to a HLA (MHC) class I restricted antigen (see Soto et al., Cancer Immunol Immunother. 62: 359-369, 2013).

In certain embodiments, a host T cell transfected to express a fusion protein of this disclosure also expresses a recombinant TCR specific to a cancer antigen. In some embodiments, the cancer antigen is a WT1. “WT1” refers to Wilm's tumor 1, a transcription factor that contains four zinc-finger motifs at the C-terminus and a proline/glutamine-rich DNA binding domain at the N-terminus. WT1 has an essential role in the normal development of the urogenital system and is mutated in a small subset of patients with Wilm's tumors. High expression of WT1 has been observed in various cancers, including, breast cancer, ovarian cancer, acute leukemias, vascular neoplasms, melanomas, colon cancer, lung cancer, thyroid cancer, bone and soft tissue sarcoma, and esophageal cancer. Alternative splicing has been noted for WT1.

In certain embodiments, a host T cell transfected to express a fusion protein of this disclosure also expresses a recombinant TCR specific to mesothelin. “Mesothelin” (MSLN) refers to a gene that encodes a precursor protein that is cleaved into two products, megakaryocyte potentiating factor and mesothelin. Megakaryocyte potentiation factor functions as a cytokine that can stimulate colony formation in bone marrow megakaryocytes. Mesothelin is a glycosylphosphatidylinositol-anchored cell-surface protein that may function as a cell adhesion protein. This protein is overexpressed in epithelial mesotheliomas, ovarian cancers and in specific squamous cell carcinomas. Alternative splicing results in multiple transcript variants.

In certain embodiments, a host T cell transfected to express a fusion protein of this disclosure also expresses a recombinant TCR specific to cyclin-A1.

In certain embodiments, a host T cell transfected to express a fusion protein of this disclosure also expresses a CAR.

### Uses

Diseases that may be treated with cells expressing fusion proteins as described in the present disclosure include cancer, infectious diseases (viral, bacterial, protozoan infections), and immune diseases (e.g., autoimmune). Adoptive immune and gene therapy are promising treatments for various types of cancer (Morgan et al., Science 314:126, 2006; Schmitt et al., Hum. Gene Ther. 20:1240, 2009; June, J. Clin. Invest. 117:1466, 2007) and infectious disease (Kitchen et al., PLoS One 4:38208, 2009; Rossi et al., Nat. Biotechnol. 25:1444, 2007; Zhang et al., PLoS Pathog. 6:e1001018, 2010; Luo et al., J. Mol. Med. 89:903, 2011).

In certain embodiments the methods provided herein are for treating a hyperproliferative disorder that is a hematological malignancy or a solid cancer. For example, the hematological malignancy to be treated may be acute lymphoblastic leukemia (ALL), acute myeloid leukemia (AML), chronic myelogenous leukemia (CIVIL), chronic eosinophilic leukemia (CEL), myelodysplastic syndrome (MDS), non-Hodgkin's lymphoma (NHL), or multiple myeloma (MM). Exemplary solid cancer to be treated may be biliary cancer, bladder cancer, bone and soft tissue carcinoma, brain tumor, breast cancer, cervical cancer, colon cancer, colorectal adenocarcinoma, colorectal cancer, desmoid tumor, embryonal cancer, endometrial cancer, esophageal cancer, gastric cancer, gastric adenocarcinoma, glioblastoma multiforme, gynecological tumor, head and neck squamous cell carcinoma, hepatic cancer, lung cancer, malignant melanoma, osteosarcoma, ovarian cancer, pancreatic cancer, pancreatic ductal adenocarcinoma, primary astrocytic tumor, primary thyroid cancer, prostate cancer, renal cancer, renal cell carcinoma, rhabdomyosarcoma, skin cancer, soft tissue sarcoma, testicular germ-cell tumor, urothelial cancer, uterine sarcoma, or uterine cancer.

Other exemplary types of cancer that may be treated include adenocarcinoma of the breast, prostate, and colon; all forms of bronchogenic carcinoma of the lung; myeloid leukemia; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, Merkel cell, mucinous, non-small cell lung, oat cell, papillary, scirrhous, bronchiolar, bronchogenic, squamous cell, and transitional cell). Additional types of cancers that may be treated include histiocytic disorders; malignant histiocytosis; leukemia; Hodgkin's disease; immunoproliferative small; non-Hodgkin's lymphoma; plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment: adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin. The types of cancers that may be treated also include angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

Exemplifying the variety of hyperproliferative disorders amenable to a fusion protein T cell therapy are B-cell cancers, including B-cell lymphomas (such as various forms of Hodgkin's disease, non-Hodgkins lymphoma (NHL) or central nervous system lymphomas), leukemias (such as acute lymphoblastic leukemia (ALL), chronic lymphocytic leukemia (CLL), Hairy cell leukemia, B cell blast transformation of chronic myeloid leukemia) and myelomas (such as multiple myeloma). Additional B cell cancers include small lymphocytic lymphoma, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt's lymphoma/leukemia, B-cell proliferations of uncertain malignant potential, lymphomatoid granulomatosis, and post-transplant lymphoproliferative disorder.

Inflammatory and autoimmune diseases include arthritis, rheumatoid arthritis, juvenile rheumatoid arthritis, osteoarthritis, polychondritis, psoriatic arthritis, psoriasis, dermatitis, polymyositis/dermatomyositis, inclusion body myositis, inflammatory myositis, toxic epidermal necrolysis, systemic scleroderma and sclerosis, CREST syndrome, inflammatory bowel disease, Crohn's disease, ulcerative colitis, respiratory distress syndrome, adult respiratory distress syndrome (ARDS), meningitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), subacute cutaneous lupus erythematosus, discoid lupus, lupus myelitis, lupus cerebritis, juvenile onset diabetes, multiple sclerosis, allergic encephalomyelitis, neuromyelitis optica, rheumatic fever, Sydenham's chorea, immune responses associated with acute and delayed hypersensitivity mediated by cytokines and T-lymphocytes, tuberculosis, sarcoidosis, granulomatosis including Wegener's granulomatosis and Churg-Strauss disease, agranulocytosis, vasculitis (including hypersensitivity vasculitis/angiitis, ANCA and rheumatoid vasculitis), aplastic anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, central nervous system (CNS) inflammatory disorders, multiple organ injury syndrome, myasthenia gravis, antigen-antibody complex mediated diseases, anti-glomerular basement membrane disease, anti-phospholipid antibody syndrome, allergic neuritis, Behcet disease, Castleman's syndrome, Goodpasture's syndrome, Lambert-Eaton Myasthenic Syndrome, Reynaud's syndrome, Sjorgen's syndrome, Stevens-Johnson syndrome, solid organ transplant rejection, graft versus host disease (GVHD), bullous pemphigoid, pemphigus, autoimmune polyendocrinopathies, seronegative spondyloarthropathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic throbocytopenic purpura (TTP), Henoch-Schonlein purpura, autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM) and Sheehan's syndrome; autoimmune hepatitis, lymphoid interstitial pneumonitis (HIV), bronchiolitis obliterans (non-transplant), non-specific interstitial pneumonia (NSIP), Guillain-Barré Syndrome, large vessel vasculitis (including polymyalgia rheumatica and giant cell (Takayasu's) arteritis), medium vessel vasculitis (including Kawasaki's disease and polyarteritis nodosa), polyarteritis nodosa (PAN) ankylosing spondylitis, Berger's disease (IgA nephropathy), rapidly progressive glomerulonephritis, primary biliary cirrhosis, Celiac sprue (gluten enteropathy), cryoglobulinemia, cryoglobulinemia associated with hepatitis, amyotrophic lateral sclerosis (ALS), coronary artery disease, familial Mediterranean fever, microscopic polyangiitis, Cogan's syndrome, Whiskott-Aldrich syndrome and thromboangiitis obliterans.

In particular embodiments, a method of treating a subject with the fusion protein as disclosed herein include acute myelocytic leukemia, acute lymphocytic leukemia, and chronic myelocytic leukemia.

Infectious diseases include those associated with infectious agents and include any of a variety of bacteria (e.g., pathogenic E. coli, S. typhimurium, P. aeruginosa, B. anthracis, C. botulinum, C. difficile, C. perfringens, H. pylori, V. cholerae, Listeria spp., Rickettsia spp., Chlamydia spp., and the like), mycobacteria, and parasites (including any known parasitic member of the Protozoa). Infectious viruses include eukaryotic viruses, such as adenovirus, bunyavirus, herpesvirus, papovavirus, papillomavirus (e.g., HPV), paramyxovirus, picornavirus, rhabdovirus (e.g., Rabies), orthomyxovirus (e.g., influenza), poxvirus (e.g., Vaccinia), reovirus, retrovirus, lentivirus (e.g., HIV), flavivirus (e.g., HCV, HBV) or the like. In certain embodiments, infection with cytosolic pathogens whose antigens are processed and displayed with HLA (MHC) Class I molecules, are treated with fusion proteins of this disclosure.

A fusion protein of this disclosure may be administered to a subject in cell-bound form (e.g., gene therapy of target cell population (mature T cells (e.g., CD8⁺ or CD4⁺ T cells) or other cells of T cell lineage)). In a particular embodiment, cells of T cell lineage expressing fusion proteins administered to a subject are syngeneic, allogeneic, or autologous cells.

Pharmaceutical compositions including fusion proteins of this disclosure may be administered in a manner appropriate to the disease or condition to be treated (or prevented) as determined by persons skilled in the medical art. An appropriate dose, suitable duration, and frequency of administration of the compositions will be determined by such factors as the condition of the patient, size, type and severity of the disease, particular form of the active ingredient, and the method of administration. The present disclosure provides pharmaceutical compositions comprising cells expressing a fusion protein as disclosed herein and a pharmaceutically acceptable carrier, diluents, or excipient. Suitable excipients include water, saline, dextrose, glycerol, or the like and combinations thereof.

In some embodiments, the disclosure is directed to a method of increasing the activity of an immune cell, enhancing or prolonging an immune response, stimulating an antigen-specific T cell response, inhibiting an immunosuppressive signaling pathway, treating cancer or a tumor, inhibiting immune resistance of cancer cells, or treating an infection, comprising administering to a subject in need thereof an effective amount of a host cell expressing a fusion protein as described herein. In further embodiments, a host cell for use in any of the aforementioned methods further expresses an engineered antigen-specific TCR, an engineered antigen-specific high affinity TCR, a CAR, a co-stimulatory molecule, or any combination thereof. In particular embodiments, methods of treating leukemia are provided, comprising co-expressing a fusion protein as disclosed herein and a recombinant, antigen-specific TCR.

In some embodiments, there are provided methods of inducing or enhancing a Class I HLA response by a CD4+ T cell, comprising administering to a subject in need thereof an effective amount of a CD4+ T cell expressing a fusion protein as described herein. In further embodiments, a host cell for use in inducing or enhancing a Class I HLA response by a CD4+ T cell further expresses an engineered antigen-specific TCR, an engineered antigen-specific high affinity TCR, a CAR, a co-stimulatory molecule, or any combination thereof.

In any of the aforementioned embodiments, the methods are effective in the absence of administering exogenous IL-2.

In still other embodiments, a subject of any of the aforementioned methods is further treated with an adjunctive therapy, such as a chemotherapy. Exemplary chemotherapeutic agents include, for example, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSKTM; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (Taxol™, Bristol-Myers Squibb Oncology, Princeton, N.J.) and docetaxel (Taxotere™, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins, capecitabine; and pharmaceutically acceptable salts, acids or derivatives of any of the above.

In some embodiments, the adjunctive therapy is a vaccine, an inhibitor of an immunosuppression signal, a B-Raf inhibitor, a MEK inhibitor, a tyrosine kinase inhibitor, a cytotoxic agent, a chemotherapeutic, or any combination thereof. In some embodiments, the inhibitor of an immunosuppression signal is an antibody or siRNA. In some embodiments, the antibody or siRNA is specific for PD-1, PD-L1, PD-L2, CTLA4, LAG3, KIR, CD244, B7-H3, B7-H4, BTLA, HVEM, GAL9, TIM3, A2aR, or any combination thereof.

### EXAMPLES

### Example 1

### Cytokine and Chemokine Profile in Carcinoma Cells

The soluble factors expressed by purified pancreas carcinoma cells in culture were profiled by quantitative PCR. Briefly, total RNA was extracted (RNeasy Miniprep Kit, Qiagen) from primary cultures of KPC tumor epithelial cells and paired metastatic cells to the livers of the same animals (n=3 each), and also from preinvasive pancreatic ductal epithelial cells. RNA was converted to cDNA using a High Capacity Reverse Transcriptase Kit (Applied Biosystems). Quantitative PCR was performed using SYBR Green mastermix and triplicate samples were run on a C1000 Thermal Cycler (BioRad). Primers were based on published literature or designed using Primer-BLAST software. Quantifications were normalized to endogenous cycA and fold-change gene expression in invasive and metastatic cells compared to preinvasive cells was calculated using the AACT method. The genetically-engineered Kras^{LSL-G12D/+}; Trp53^{LSL-R172H/+}; Cre (KPC) mouse model of PDA was previously described by Hingorani et al. (Cancer Cell 7:469, 2005).

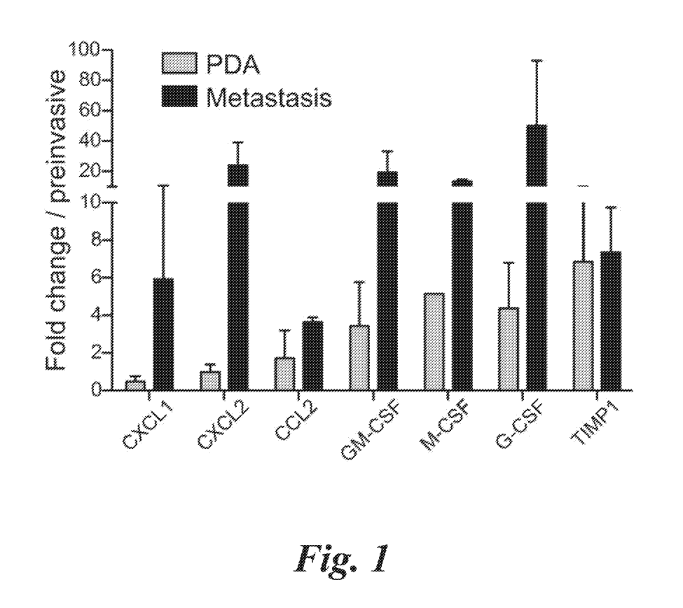

The secretory profile of invasive and metastatic cells revealed a substantial commitment to synthesis of growth factors and chemokines involved in both granulocyte (e.g., CXCL1, CXCL2) and monocyte (e.g., CCL2) trafficking (data not shown). Granulocyte macrophage colony stimulating factor (GM-CSF), granulocyte colony stimulating factor (G-CSF), and monocyte colony stimulating factor (M-CSF) were also upregulated in tumor epithelial cells as compared to preinvasive ductal cells. Relative gene expression by quantitative PCR confirmed the increase in these factors in invasive versus preinvasive cells (FIG. 1, see Stromnes et al., Gut 63:1769, 2014). Of these three myelopoietic cytokines, GM-CSF had the most pronounced effect on promoting granulocytic myeloid-derived suppressor cell (MDSC) survival in vitro (data not shown).

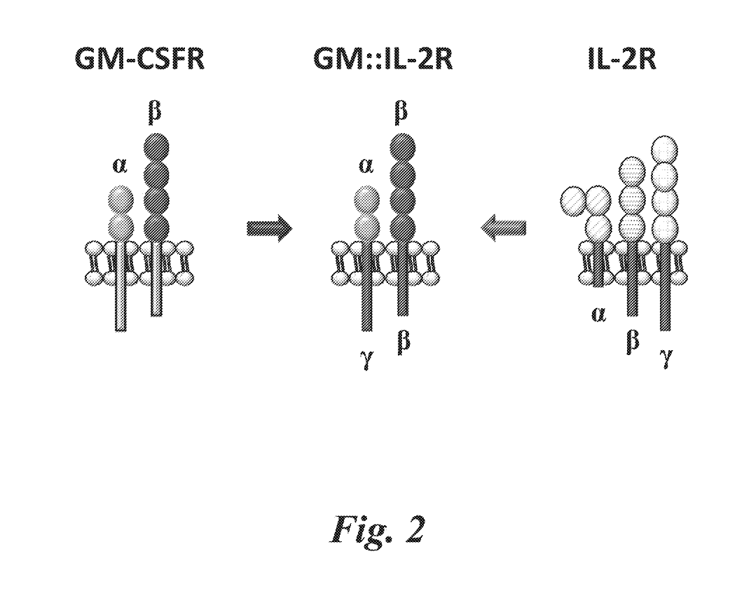

As described herein, embodiments provided herein, such as fusion proteins and complexes, are based in some aspect on these and/or related features of tumor microenvironments, such as environments of metastatic or invasive tumors. In some embodiments, provided are compositions and methods to enhance anti-tumor activity of immune therapies (e.g., an exemplary fusion protein comprised of extracellular components from CSF2R (GM-CSFR) and intracellular components from IL-2R is illustrated in FIG. 2).

### Example 2

### Generation of High Affinity Mesothelin-Specific TCRs

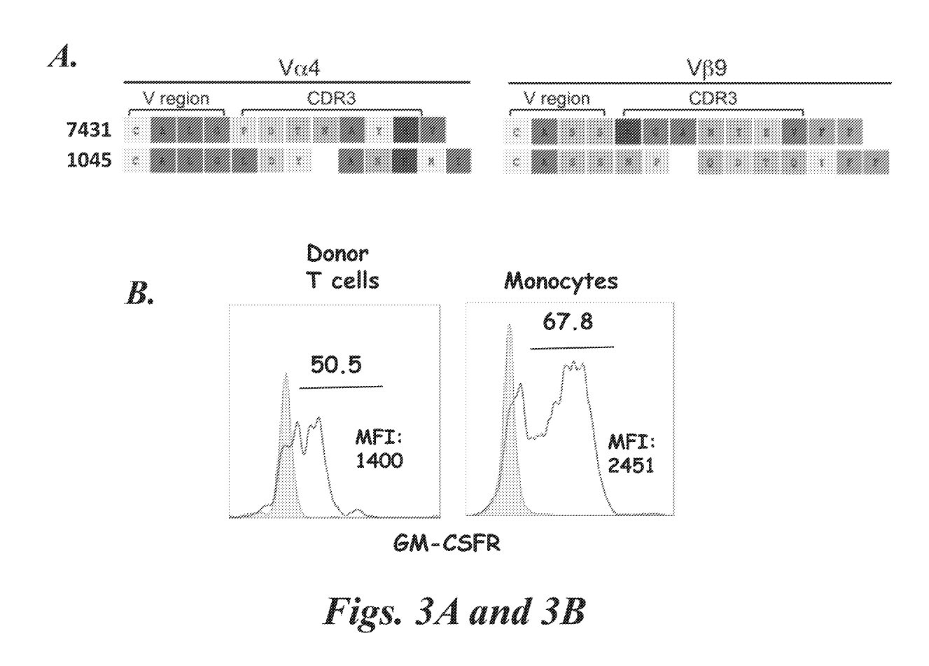

B6 Msln^{-/-} and wild-type (WT) mice were immunized with a recombinant adenovirus expressing murine Msln (Ad-Msln) to elicit reactive T cells. T cells specific for epitopes Msln343-351, Msln484-492, Msln544-552, and Msln583-591 were isolated from Msln^{-/-} mice, but not WT mice, consistent with central tolerance (Stromnes et al., Cancer Cell 28:638, 2015). However, both Msln^{-/-} and WT mice generated responses to Msln₄₀₆₋₄₁₄, previously shown to be processed and presented by a B6 ovarian cancer cell line (Hung et al., Gene Ther. 4:921,2007). Msln₄₀₆₋₄₁₄-specific T cells isolated from WT mice uniformly expressed the Vβ9 TCR chain, as did the majority of Msln₄₀₆₋₄₁₄-specific T cells from Msln^{-/-} mice (Stromnes et al., 2015). Despite expressing similar levels of Vβ9, Msln₄₀₆₋₄₁₄-specific T cell lines from Msln^{-/-} mice stained brighter with tetramer, consistent with higher affinity (Stromnes et al., 2015). Msln^{-/-} Msln₄₀₆₋₄₁₄-specific T cell clones also responded to lower antigen concentrations than the corresponding WT clones (Stromnes et al., 2015). Most T cell clones isolated from WT and Msln^{-/-} mice used the same germline Vα4 and Vβ9 TCR chains, restricting any sequence differences between the highest affinity clones from the respective strains to CDR3 (FIG. 3A).

### Example 3

### Assesment of Exemplary CSF2R::IL-2R Constructs in Mouse Model

Exemplary CSF2R::IL-2R chimeric constructs, and impacts thereof on function of antigen-specific engineered T cells, were assessed in a preclinical mouse model for disseminated leukemia, based on the murine C57BL/6 Friend virus-induced erythroleukemia (FBL) and TCR_{gag} transgenic mice.

CSF2R::IL-2R chimeric constructs based on murine genes (similar to those illustrated in FIG. 2) and/or a mesothelin-targeted T cell receptor (TCR₁₀₄₅ (MSLN₄₀₆₋₄₁₄ specific))-encoding construct (Stromnes et al., Cancer Cell 28:638, 2015, wherein TCR₁₀₄₅ (including sequence) is incorporated herein by reference) were inserted into the pMP71 retroviral vector and used to transduce primary P14 Thy1.1⁺ mouse splenocytes stimulated with anti-CD3 and anti-CD28 antibodies. Fusion protein constructs were generated by PCR. The constructs were then directionally TOPO-cloned into the pENTR™/D-TOPO® vector (Invitrogen), and transferred into the retroviral vector pMP71-attR using Gateway® technology (Invitrogen). The retroviral packaging cell line Plat-E (Morita et al., 2000, Gene Therapy 7:1063-1066, 2000; Cell Biolabs, Inc.) was transduced with the retroviral vector using effectene transduction reagent (Qiagen). Viral supernatant was collected on days 2 and 3 post-transfection and then used to transduce T cells, in some cases containing TCR₁₀₄₅. One day prior to transfection, P14 Thy1.1⁺ T cells were stimulated with anti-CD3/CD28 and 100 U/mL rhlL-2. Transduction of P14 Thy1.1⁺ T cells was performed in 12 well plates in the presence of IL-2 and polybrene by spinfection for 90 minutes at 1000 g. TCR₁₀₄₅ transduced T cells were restimulated with irradiated Thy.12⁺ splenocytes pulsed with Msln406-414 peptide (GQKMNAQAI, 1 μg/ml) and recombinant human IL-2 (r-IL2, 50 IU/ml) seven days following T cell activation with anti-CD3/CD28. On day 5 after antigen re-stimulation, >90% T cells expressed the introduced TCR. 5×10⁶ cells expressing TCR₁₀₄₅ were infused into Thy1.2⁺ C57BL/6 (B6) mice (Jackson Laboratory) together with 5×10⁸ pfu of a recombinant attenuated adenovirus vaccine (i.m.) engineered to express recombinant murine mesothelin (Ad-Msln). The infused TCR₁₀₄₅+ donor cells included a population expressing the GM/IL2R fusion protein (“GM/IL2R”) and a population not expressing the construct (“WT”). Donor cells were tracked on days 0, 8, 14, and 21 after infusion.

In more detail, TCR₁₀₄₅⁺ donor (Thy1.1⁺/Vβ9⁺-gated) T cells were analyzed by flow cytometry for surface expression of a molecule containing the extracellular portion of a GM-CSFR (with expression on monocytes used as a positive control). At day 0, approximately 50% of the TCR₁₀₄₅⁺ donor T cells were observed by this assay to express the chimeric molecule (FIG. 3B).

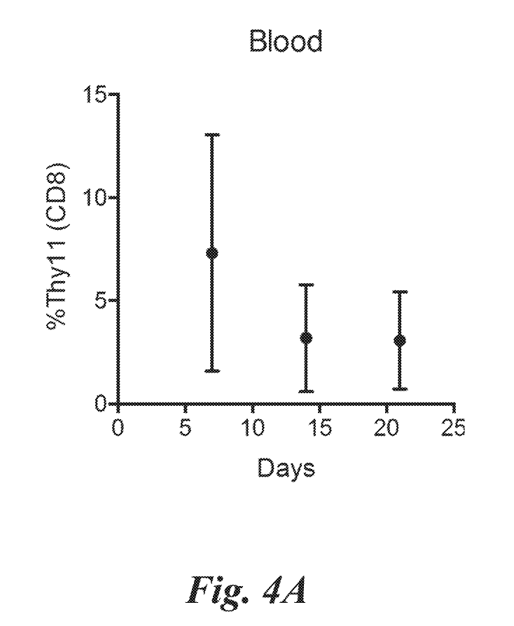

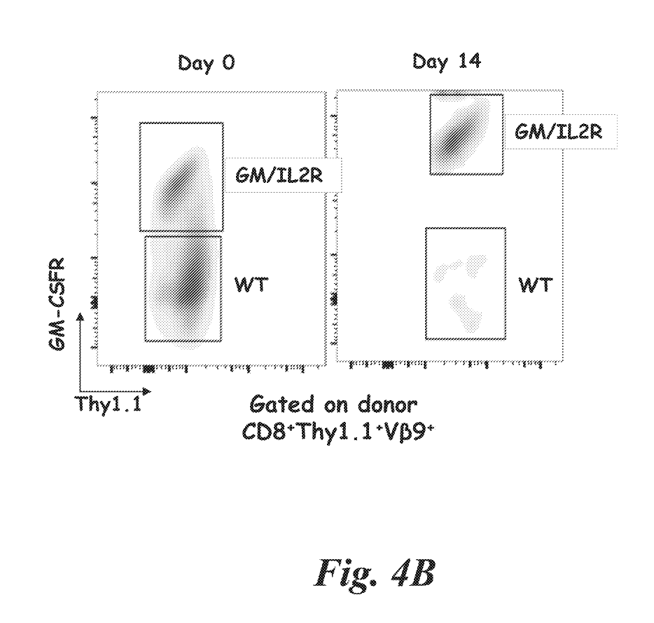

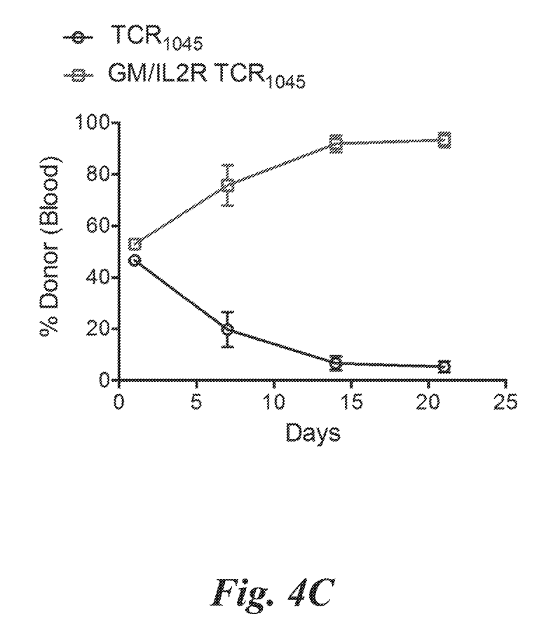

The presence of donor T cells in the blood of the animals continued to be monitored over time. Donor T cells persisted and were detectable in the blood for at least 21 days following transfer (FIG. 4A).

As shown in FIGS. 4B and 4C, donor T cells expressing the CSF2R::IL-2R fusion protein (as determined by anti-GM-CSF staining) (“GM/IL2R”) exhibited a survival and/or proliferative advantage, as compared to donor T cells that did not express the fusion protein (“WT”). For example, by day 14 post-transfer and continuing through day 21, GM/IL2R-expressing cells represented nearly all of the donor T cells detected in the blood (FIGS. 4B and 4C). These results indicate that the fusion protein provided a persistence advantage to adoptively transferred T cells expressing TCR₁₀₄₅.

These data indicate that fusion proteins of this disclosure in some embodiments provide T cells, such as T cells containing antigen-specific TCRs, a survival and/or expansion advantage, which is consistent with utility of the construct to improve persistence and exposure to transferred cells, including improving efficacy in a tumor microenvironment.

All of the U.S. patents, U.S. patent application publications, U.S. patent applications, foreign patents, foreign patent applications, and non-patent publications referred to in this specification or listed in the Application Data Sheet, including but not limited to, U.S. Provisional Patent Application No. 62/325,428, filed Apr. 20, 2016, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A host cell, comprising a fusion protein and an antigen binding protein,
wherein the fusion protein comprises a transmembrane domain disposed between an extracellular component comprising a cytokine binding domain or portion thereof, and an intracellular component comprising an IL-2R intracellular portion, intracellular signaling domain or a portion thereof; and
wherein the antigen binding protein is a T cell receptor (TCR); a chimeric antigen receptor (CAR); or optionally a plurality of antigen binding proteins, e.g., a TCR and a CAR.

2. A host cell, comprising a first fusion protein, a second fusion protein and optionally an antigen binding protein,
wherein the first fusion protein comprises a transmembrane domain disposed between an extracellular component comprising a cytokine binding domain or portion thereof, and an intracellular component that is comprised of, or has at least 90% identity to, an IL-2Rγ, optionally human IL-2Rγ, intracellular portion or intracellular signaling domain or portion thereof, or optionally has at least 90% identity to SEQ ID NO.:10;
wherein the second fusion protein comprises a transmembrane domain disposed between an extracellular component comprising a cytokine binding domain or portion thereof, and an intracellular component is comprised of, or has at least 90% identity to, an IL-2Rβ, optionally human IL-2Rβ, intracellular signaling domain or portion thereof and/or a signaling domain or portion thereof of an IL-4R, IL-7R, IL-9R, IL-15R or IL-21R chain, or optionally has at least 90% identity to SEQ ID NO.:12; and
wherein the optional antigen binding protein comprises a T cell receptor (TCR); a chimeric antigen receptor (CAR); and optionally comprises a plurality of antigen binding proteins, e.g., a TCR and a CAR.

3. The host cell of claim 1 or 2, wherein the extracellular component and/or the extracellular component of the first and/or second fusion protein comprises an extracellular portion of a CSF2R, CSF1R, CSF3R, CXCR2, or CCR8, optionally of a human CSF2R, CSF1R, CSF3R, CXCR2, or CCR8.

4. The host cell of any one of the preceding claims, wherein the cytokine binding domain of the fusion protein specifically binds to, or contains at least a portion of the binding site for, a GM-CSF, M-CSF, G-CSF, CXCL1, CXCL2, or CCL1, and optionally comprises or consists of a human sequence.

5. The host cell of any one of the preceding claims, wherein the cytokine binding domain is a GM-CSF binding domain and/or is from, or has at least 90% identity to, an extracellular portion of a CSF2R, which optionally is a human CSF2R, or optionally has at least 90% identity to an extracellular portion of a sequence put forth in SEQ ID NO.:9 or SEQ ID NO.:11.

6. The host cell of any one of the preceding claims, wherein the transmembrane domain of the fusion protein and/or of the first and/or second fusion protein, comprises a transmembrane domain of an IL-2RG, IL-2RB, IL-2RA, IL-4R, IL-7R, IL-9R, IL-15R or IL-21R, optionally of human origin.

7. The host cell of any one of the preceding claims, wherein the transmembrane domain of the fusion protein comprises a transmembrane domain of a CSF2RA, CSF2RB, CSF1R, CSF3R, CXCR2, or CCR8, optionally of human origin, or optionally has at least 90% amino acid sequence identity to SEQ ID NO.:22 or 23.

8. The host cell of any one of the preceding claims, wherein the transmembrane domain comprises a transmembrane domain of a CD2, CD3ε, CD3δ, CD3ζ, CD25, CD27, CD28, CD40, CD79A, CD79B, CD80, CD86, CD95 (Fas), CD134 (OX40), CD137 (4-1BB), CD150 (SLAMF1), CD152 (CTLA4), CD200R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GAL9, KIR, Lck, LAT, LRP, NKG2D, NOTCH1, NOTCH2, NOTCH3, NOTCH4, PTCH2, ROR1, ROR2, Ryk, Slp76, SIRPα, pTα, TCRα, TCRβ, TIM3, TRIM, LPA5, or Zap70, optionally of human origin.

9. The host cell of any one of the preceding claims, wherein the host cell comprises at least two fusion proteins that are capable of associating to form a heteromultimer on the host cell surface.

10. The host cell of claim 9, wherein the fusion proteins each comprise a different extracellular component, wherein the different extracellular components are capable of associating with each other to form a functional cytokine binding domain.

11. The host cell of claim 10, wherein one of the different extracellular components is comprised of, or has at least 90% identity to, a CSF2Rα, optionally human CSF2Rα, extracellular portion, extracellular cytokine binding domain or portion thereof, or optionally has at least 90% identity to SEQ ID NO.:9, and the other different extracellular component is comprised of, or has at least 90% identity to, a CSF2Rβ, optionally human CSF2Rβ, extracellular portion, extracellular cytokine binding domain or portion thereof, or optionally has at least 90% identity to SEQ ID NO.:11.

12. The host cell of any one of claims 9-11, wherein the fusion proteins each comprise a different intracellular component, wherein the different intracellular components are capable of associating with each other to form a functional intracellular signaling domain.

13. The host cell of claim 12, wherein at least one of the different intracellular components is comprised of, or has at least 90% identity to, an IL-2Rγ intracellular portion, intracellular signaling domain or portions thereof, or optionally has at least 90% identity to SEQ ID NO.:10.

14. The host cell of claim 12 or 13, wherein at least one of the different intracellular components is comprised of, or has at least 90% identity to, an IL-2Rβ, IL-4RA, IL-7R, IL-15RA, or IL-21R intracellular portion, intracellular signaling domain or portions thereof, which in each case, individually, is optionally human-derived, or optionally has at least 90% identity to SEQ ID NO.:12.

15. The host cell of any one of claims 9-14, wherein the heteromultimer on the host cell surface is a heterodimer or heterotrimer.

16. The host cell of claim 15, wherein the one fusion protein consists of, comprises, or has at least 90%, at least 95%, at least 96%, at least 97%, at least 98%, at least 98%, or at least 99% identity to SEQ ID NO.:1 and the other fusion protein consists of, comprises, or has at least 90%%, at least 95%, at least 96%, at least 97%, at least 98%, at least 98%, or at least 99% identity to SEQ ID NO.:2.

17. The host cell of any one of the preceding claims, wherein the antigen binding protein is a T cell receptor (TCR), or optionally is an antigen-specific TCR.

18. The host cell of any one of the preceding claims, wherein the antigen-specific TCR is exogenous to the host cell and/or a host to whom the host cell will be administered.

19. The host cell of claim 17 or 18, wherein the TCR binds to an antigen::HLA complex with high affinity.

20. The host cell of claim 19, wherein the high affinity binding has a Kₐ equal to or greater than 10⁷M⁻¹.

21. The host cell of any one of claims 17-20, wherein the TCR is specific to a HLA class I restricted antigen.

22. The host cell of any one of the preceding claims, wherein the antigen is a cancer-specific antigen.

23. The host cell of claim 22, wherein the cancer-specific antigen comprises WT-1, mesothelin, ROR1 or cyclin-A1.

24. The host cell of any one of the preceding claims, wherein the TCR is WT-1 specific TCR designated as C4.

25. The host cell of any one of the preceding claims, wherein the antigen binding protein is a CAR.

26. The host cell of claim 25, wherein the chimeric antigen receptor comprises an extracellular antigen binding domain and an intracellular signaling domain capable of delivering a primary signal to a T cell and optionally a costimulatory domain.

27. The host cell of claim 26, wherein the intracellular signaling domain comprises an intracellular signaling domain of a costimulatory molecule.

28. The host cell of claim 27, wherein the costimulatory molecule comprises CD28, CD137 (4-1BB), or ICOS.

29. The host cell of any one of claims 26-28, wherein the intracellular signaling domain comprises an intracellular signaling domain of a CD3ε, CD3δ, CD3ζ, CD25, CD27, CD28, CD40, CD47, CD79A, CD79B, CD134 (OX40), CD137 (4-1BB), CD150 (SLAMF1), CD278 (ICOS), CD357 (GITR), CARD11, DAP10, DAP12, FcRα, FcRβ, FcRγ, Fyn, Lck, LAT, LRP, NKG2D, NOTCH1, NOTCH2, NOTCH3, NOTCH4, ROR2, Ryk, Slp76, pTα, TCRα, TCRβ, TRIM, Zap70, PTCH2, or any combination thereof and/or wherein the intracellular signaling portion of the chimeric antigen receptor comprises a primary activation signaling domain, which optionally is derived from CD3 ζ, and does not comprise a costimulatory domain and/or does not comprise a CD28 signaling domain, a 4-1BB signaling domain and/or an ICOS signaling domain.

30. The host cell of any one of claims 26-29, wherein the intracellular signaling domain comprises a costimulatory domain of a CD137 (4-1BB), CD27, CD28, ICOS, OX40 (CD134), or any combination thereof.

31. The host cell of any one of claims 26-30, wherein the intracellular signaling domain comprises a costimulatory domain of a CD137 (4-1BB) or CD28, or any combination thereof.

32. The host cell of any one of claims 26-31, wherein the intracellular signaling domain comprises a costimulatory domain of a CD28.

33. The host cell of any one of claims 26-32, wherein the intracellular signaling domain comprises a costimulatory domain of a CD137 (4-1BB).

34. The host cell of any one of claims 26-33, wherein the intracellular signaling domain comprises a second intracellular signaling domain.

35. The host cell of claim 34, wherein the second intracellular signaling domain comprises an intracellular signaling domain of a CD137 (4-1BB).

36. The host cell of any one of claims 26-33, wherein the CAR antigen binding domain is an antibody binding fragment or scFv specific for the antigen.

37. The host cell of any one of the preceding claims, wherein the host cell comprises at least two antigen binding proteins, wherein the at least two antigen binding proteins include a TCR and a CAR.

38. The host cell of any of claims 1-37, wherein the expression of the fusion protein in a T cell comprising a TCR or chimeric antigen receptor specific for an antigen results in at least about a 1.5-fold, 2-fold, or 3-fold increase in survival, expansion, cytotoxicity, cytokine secretion, and/or response to multiple rounds of stimulation, by the T cell, in response to binding of the antigen and/or following administration to a subject, and/or results in at least about a 1.5-fold, 2-fold, or 3-fold increase in time of survival, disease-free survival, or amelioration of one or more disease symptom, of a subject to which the cell is administered, as compared to a cell substantially the same as the T cell but not containing the fusion protein.

39. The host cell of any one of the preceding claims, wherein the host cell is an immune system cell.

40. The host cell of claim 39, wherein the immune system cell is a T cell.

41. The host cell of claim 40, wherein the T cell is a CD4+ T cell.

42. The host cell of claim 40, wherein the T cell is a CD8+ T cell.

43. A host cell, comprising a nucleic acid molecule encoding a fusion protein and an antigen binding protein of any one of claims 1-42.

44. A host cell, comprising a vector, wherein the vector comprises a nucleic acid molecule encoding a fusion protein and an antigen binding protein of any one of claims 1-42.

45. The host cell of claim 44, wherein the vector is a viral vector.

46. The host cell of claim 45, wherein the viral vector is a lentiviral or retroviral vector.

47. The host cell of claim 46, wherein the viral vector is a lentiviral vector.

48. The host cell of any one of the preceding claims, wherein the TCR is an antigen-specific TCR.

49. A method of treating a disease in a subject, comprising administering a host cell according to any one of claims 1-48, and/or a fusion protein, nucleic acid, vector, composition, or complex according to any of claims 62-66, to the subject.

50. The method of claim 49, wherein the disease is selected from the group consisting of viral infection, bacterial infection, cancer, and autoimmune disease.

51. The method of claim 50, wherein the disease is cancer.

52. The method of claim 51, wherein the cancer is a tumor.

53. The method of claim 52, wherein the tumor is a hematologic tumor, which is optionally CLL or MCL, and/or is a solid tumor, which optionally is a breast cancer, lung cancer, ovarian cancer, or pancreatic cancer tumor, which optionally is a lung adenocarcinoma, adenocarcinoma, squamous cell carcinoma, small cell carcinoma, atypical carcinoid, or triple-negative breast cancer.

54. The method of claim 52 or 53, wherein the tumor comprises a primary tumor, a metastatic tumor, or both.

55. The method of any one of claims 52-54, wherein the tumor cells overexpress WT-1, mesothelin, ROR1 or cyclin-A1.

56. The method of any one of claims 49-55, wherein the subject is human.

57. A host cell of any one of claims 1-48, for use in treating a viral infection, bacterial infection, cancer, or autoimmune disease.

58. A fusion protein, comprising an extracellular component comprising all or a portion of a cytokine binding domain, a transmembrane domain and an intracellular component comprising a signaling domain of one or more IL-2R chain or signaling portion(s) thereof, wherein the cytokine binding domain is not an IL-2 binding domain.

59. A nucleic acid comprising a nucleotide sequence encoding the fusion protein of claim 58, optionally wherein fusion protein is encoded by a polynucleotide comprising, consisting of or having at least 80%, at least 85%, at least 90%, at least 95%, or at least 98% identity to the polynucleotide sequence of SEQ ID NO.:6 or 7.

60. The nucleic acid of claim 59, wherein (a) the fusion protein is a first fusion protein and the all or portion of the cytokine binding domain is a first portion of the cytokine binding domain and (b) the nucleic acid further comprises a nucleotide sequence encoding a second fusion protein, the second fusion protein comprising a second portion of the cytokine binding domain, a second transmembrane domain and a second intracellular component comprising a second IL-2R chain signaling domain or signaling portion thereof and optionally further comprising a nucleotide sequence encoding an antigen receptor or portion thereof.

61. A vector, comprising the nucleic acid of claim 59 or 60.

62. A composition, comprising the nucleic acid of claim 59 and an additional nucleic acid, wherein (a) the fusion protein is a first fusion protein and the all or portion of the cytokine binding domain is a first portion of the cytokine binding domain and (b) the additional nucleic acid comprises a nucleotide sequence encoding a second fusion protein, the second fusion protein comprising a second portion of the cytokine binding domain, a second transmembrane domain and a second intracellular component comprising a second IL-2R chain signaling domain or signaling portion thereof, wherein the nucleic acid and additional nucleic acids are optionally expressed from the same or different vectors, and optionally further comprising a nucleic acid encoding an antigen receptor or portion thereof.

63. A molecular complex, comprising the fusion protein of claim 58, which is a first fusion protein, and further comprising a second fusion protein, the second fusion protein comprising a second portion of the cytokine binding domain, a second transmembrane domain and a second intracellular component comprising a second IL-2R chain signaling domain or signaling portion thereof, wherein the first and second fusion proteins are present in a complex, via covalent or non-covalent interaction.

64. The fusion protein, nucleic acid, vector, composition, or complex of any of claims 58-63, wherein the first and/or second fusion protein comprises an extracellular component of a human GM-CSFR, optionally having at least 90% identity to SEQ ID NO.:9 or SEQ ID NO.:11, and an intracellular signaling portion of a human IL-2R complex chain, optionally having at least 90% identity to SEQ ID NO.:10 or SEQ ID NO.:12.

65. The fusion protein, nucleic acid, vector, composition, or complex of claim 64, wherein one of the first and/or second fusion protein comprises an extracellular component of a human CSF2Rβ chain and an intracellular signaling portion of a human IL-2Rβ chain, wherein the CSF2Rβ chain and an intracellular signaling portion of a human IL-2Rβ chain optionally have at least 90% identity to SEQ ID NO.: 11 and SEQ ID NO.: 12, respectively, and/or one of the first and/or second fusion protein comprises an extracellular component of a human CSF2Rα chain and an intracellular signaling portion of a human IL-2Rγ chain, wherein the CSF2Rβ chain and an intracellular signaling portion of a human IL-2Rβ chain optionally have at least 90% identity to SEQ ID NO.: 9 and SEQ ID NO.: 10, respectively.

66. The fusion protein, nucleic acid, vector or composition of any of claims 58-63, which encodes the fusion protein or proteins of the host cell of any of claims 1-48.
